(19) United States
(12) Patent Application Publication
(10) Pub. No.: US 2024/0287599 A1
(43) Pub. Date: Aug. 29, 2024

(54) REFERENCE FREE SPOT DECONVOLUTION IN SPATIAL TRANSCRIPTOMICS

(71) Applicant: 10x Genomics, Inc., Pleasanton, CA (US)

(72) Inventors: Stephen R. Williams, Pleasanton, CA (US); Govinda M. Kamath, Pleasanton, CA (US); Joseph Glenn Arthur, Pleasanton, CA (US)

(21) Appl. No.: 18/587,627

(22) Filed: Feb. 26, 2024

Related U.S. Application Data
(60) Provisional application No. 63/487,227, filed on Feb. 27, 2023.

(51) Int. Cl. C12Q 1/6874; C12Q 1/6841; C12Q 1/6881; G16B 20/00; G16B 40/20

(52) U.S. Cl. CPC C12Q 1/6874; C12Q 1/6881; G16B 20/00; G16B 40/20; C12Q 1/6841

(57) ABSTRACT

Systems and methods for determining cell type are provided. Information is obtained including, for each spot in a set of capture spots, a position in a tissue image and analyte abundances in the tissue. A current iteration of cell types, set to a maximum number, is determined using the analyte abundances, where each cell type has an abundance value for each analyte. When the current iteration exceeds a minimum number of cell types, a distance metric between each cell type in the current iteration is determined based on cell type analyte abundance values and the current iteration is reformed by merging a first and second cell type having a smallest distance metric. The procedure is repeated until the current iteration matches the minimum number. For each current iteration, output is determined including, for each cell type, for each spot, a proportion of cells in the spot having the cell type.

### CROSS REFERENCE TO RELATED APPLICATIONS

This application claims priority to U.S. Provisional Patent Application No. 63/487,227, filed Feb. 27, 2023, which is hereby incorporated by reference in its entirety.

### TECHNICAL FIELD

This specification describes technologies relating to reference-free determination of cell type within a tissue sample.

### BACKGROUND

Advances in single-cell and spatial transcriptomic technologies have facilitated fundamental understanding of cancers including critical cell type identification, cellular roles in tumor biology, and cell interactions in the tissue microenvironment. While both technologies have revolutionized the way in which researchers investigate cancers, they also include fundamental limitations. For instance, single-cell sequencing lacks spatial context and commercially available spatial transcriptomic products lack single-cell whole transcriptome resolution. Because of this, the integration of these modalities becomes a technical challenge for which improved tools are needed in the art to adequately address such drawbacks.

### SUMMARY

Technical solutions (e.g., computing systems, methods, and non-transitory computer readable storage mediums) for addressing the above-identified problems with determining cell type in a tissue sample are provided in the present disclosure. In particular, the present disclosure provides a reference-free spatial deconvolution approach that allows for spatial transcriptomics at increased resolution and refines estimated cell type classification over available approaches.

The following presents a summary of the present disclosure in order to provide a basic understanding of some of the aspects of the present disclosure. This summary is not an extensive overview of the present disclosure. It is not intended to identify key/critical elements of the present disclosure or to delineate the scope of the present disclosure. Its sole purpose is to present some of the concepts of the present disclosure in a simplified form as a prelude to the more detailed description that is presented later.

One aspect of the present disclosure provides a method for determining cell type. In some embodiments, the method is performed at a computer system comprising one or more processing cores and a memory. The method includes obtaining, in electronic form, input information for a set of capture spots, the input information including, for each capture spot in the set of capture spots, a corresponding position in an image of a tissue sample, and a respective abundance of each analyte in a plurality of analytes measured for each capture spot in the set of capture spots from the tissue sample. The method further includes determining a current iteration of a plurality of proposed cell types that is set to a maximum number of proposed cell types using the respective abundance of each analyte in the plurality of analytes measured for each capture spot in the set of capture spots from the tissue sample. Each respective cell type in the current iteration of the plurality of proposed cell types has an abundance value for each analyte in the plurality of analytes.

The method further includes, when the current iteration of the plurality of proposed cell types exceeds a minimum number of proposed cell types, performing a procedure comprising determining a respective distance metric between each proposed cell type in the current iteration of the plurality of proposed cell types based on the abundance value for each analyte in the plurality of analytes for each proposed cell type in the current iteration of the plurality of proposed cell types, and reforming the current iteration of the plurality of proposed cell types by merging a first proposed cell type and a second proposed cell type having a smallest distance metric among all unique pairs of proposed cell types in the current iteration of the plurality of proposed cell types. The procedure is repeated until the current iteration of the plurality of proposed cell types matches the minimum number of proposed cell types. Output information is determined, for each respective current iteration of the plurality of proposed cell types, for each respective proposed cell type in the respective current iteration of the plurality of proposed cell types, for each respective capture spot in the set of capture spots, providing a respective proportion of cells in the respective capture spot having the respective proposed cell type.

In some embodiments, the method further includes, for each respective current iteration of the plurality of proposed cell types, for each proposed cell type in the respective current iteration of the plurality of proposed cell types, providing a corresponding plurality of analytes in the proposed cell type and, for each analyte in the corresponding plurality of analytes, an abundance of the analyte.

In some embodiments, the method further includes overlaying, for each capture spot in the set of capture spots, an indication of a respective proportion of cells in the respective capture spot having a proposed cell type in the current iteration of the plurality of proposed cell types.

In some embodiments, a capture spot in the set of capture spots comprises a capture domain.

In some embodiments, a capture spot in the set of capture spots comprises a cleavage domain.

In some embodiments, each capture spot in the set of spots is attached directly or attached indirectly to a substrate.

In some embodiments, the plurality of analytes comprises five or more analytes, ten or more analytes, fifty or more analytes, one hundred or more analytes, five hundred or more analytes, 1000 or more analytes, 2000 or more analytes, or between 2000 and 100,000 analytes.

In some embodiments, each capture spot in the set of capture spots has a unique spatial barcode that encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10^{12}.}

In some embodiments, each respective capture spot in the set of capture spots includes 1000 or more capture probes, 2000 or more capture probes, 10,000 or more capture probes, 100,000 or more capture probes, 1×10⁶ or more capture probes, 2×10⁶ or more capture probes, or 5×10⁶ or more capture probes.

In some embodiments, each capture probe in the respective capture spot includes a poly-A sequence or a poly-T sequence and the unique spatial barcode that characterizes the respective capture spot.

In some embodiments, each capture probe in the respective capture spot includes the same spatial barcode from a plurality of spatial barcodes.

In some embodiments, each capture probe in the respective capture spot includes a different spatial barcode from a plurality of spatial barcodes.

In some embodiments, the tissue sample has a depth of 100 microns, 50 microns, 30 microns, 20 microns or less.

In some embodiments, a respective capture spot in the set of capture spots includes a respective plurality of capture probes, where each capture probe in the plurality of capture probes includes a capture domain that is characterized by a capture domain type in a plurality of capture domain types, and each respective capture domain type in the plurality of capture domain types is configured to bind to a different analyte in the plurality of analytes.

In some embodiments, the plurality of capture domain types comprises between 5 and 15,000 capture domain types and the respective plurality of capture probes (e.g., capture probe plurality) includes at least five, at least 10, at least 100, or at least 1000 capture probes for each capture domain type in the plurality of capture domain types.

In some embodiments, a respective capture spot in the set of capture spots includes a plurality of capture probes, where each capture probe in the plurality of capture probes includes a capture domain that is characterized by a single capture domain type configured to bind to each analyte in the plurality of analytes in an unbiased manner.

In some embodiments, each respective capture spot in the set of capture spots is contained within a 100 micron by 100 micron square on the substrate.

In some embodiments, a distance between a center of each respective capture spot to a neighboring capture spot in the set of capture spots on the substrate is between 5 microns and 100 microns.

In some embodiments, a shape of each capture spot in the set of capture spots on the substrate is a closed-form shape.

In some embodiments, the closed-form shape is circular, elliptical, or an N-gon, where N is a value between 1 and 20.

In some embodiments, the closed-form shape is circular and each capture spot in the set of capture spots has a diameter of 80 microns or less.

In some embodiments, the closed-form shape is circular and each capture spot in the set of capture spots has a diameter of between 30 microns and 65 microns.

In some embodiments, a distance between a center of each respective capture spot to a neighboring capture spot in the set of capture spots on the substrate is between 50 microns and 80 microns.

In some embodiments, the method further includes using the output information to determine whether or not the tissue sample was obtained from a subject with a condition.

In some embodiments, the method further comprises providing a treatment of the subject when it is determined that the subject has the condition.

In some embodiments, the treatment comprises a composition comprising a small molecule compound and one or more excipient and/or one or more pharmaceutically acceptable carrier and/or one or more diluent.

In some embodiments, the small molecule compound has a molecular weight of 2000 Daltons or less.

In some embodiments, the small molecule compound satisfies any two or more rules, any three or more rules, or all four rules of Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, the condition is inflammation or pain.

In some embodiments, the condition is a disease.

In some embodiments, the condition is asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersinia pestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection.

In some embodiments, the determining makes use of a latent Dirichlet allocation model.

In some embodiments, the determining refines the latent Dirichlet allocation model using expectation maximization.

In some embodiments, the method further includes, prior to the determining, removing from the plurality of analytes those analytes that are present in more than a first threshold percentage of the set of capture spots, and removing from the plurality of analytes those analytes that are present in less than a second threshold percentage of the set of capture spots.

In some embodiments, the first threshold percentage is 95% and the second threshold percentage is 5%.

In some embodiments, each analyte in the plurality of analytes is a different gene, protein, mRNA, genomic DNA, intracellular protein, metabolite, or V(D)J sequence.

In some embodiments, the set of capture spots comprises 1000 capture spots.

In some embodiments, the set of capture spots comprises 2000, 3000, 4000, 5000, 6000, 7000, 8000, 9000, or 10000 capture spots.

In some embodiments, the plurality of analytes comprises 100, 200, 300, 400, 500, or 1000 analytes.

In some embodiments, the maximum number of proposed cell types is between 8 and 40 proposed cell types.

In some embodiments, the maximum number of proposed cell types is between 5 and 100 proposed cell types.

In some embodiments, the minimum number of proposed cell types is between 2 and 10 proposed cell types.

In some embodiments, each capture spot in at least 10 percent of the set of capture spots includes analyte data from between 1 and 20 proposed cell types in the current iteration of the plurality of proposed cell types.

In some embodiments, each capture spot in at least 10 percent of the set of capture spots includes analyte data from between 2 and 10 proposed cell types in the current iteration of the plurality of proposed cell types.

In some embodiments, the plurality of analytes has higher-than-expected expression variance across the set of capture spots.

In some embodiments, the determining comprises obtaining a matrix dimensioned by a plurality of objects and a plurality of terms, where each respective object in the plurality of objects represents a corresponding capture spot in the set of capture spots, each respective term in the plurality of terms represents a corresponding analyte in the plurality of analytes, and the obtaining utilizes natural language processing to populate, for each respective object in the plurality of objects and for each respective term in the plurality of terms, a corresponding abundance for the respective analyte in the tissue sample, or a representation thereof, measured at the respective capture spot.

In some embodiments, the natural language processing comprises a generative statistical model that is further refined by a variational expectation-maximization or Markov chain Monte Carlo procedure.

Another aspect of the present disclosure provides a computer system comprising one or more processing cores and a memory, the memory storing instructions that use the one or more processing cores to perform any of the methods disclosed herein.

Another aspect of the present disclosure provides a non-transitory computer readable storage medium. The non-transitory computer readable storage medium stores instructions, which when executed by a computer system, cause the computer system to perform any of the methods disclosed herein.

As disclosed herein, any embodiment disclosed herein when applicable can be applied to any aspect.

Various embodiments of systems, methods and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The implementations disclosed herein are illustrated by way of example, and not by way of limitation, in the figures of the accompanying drawings. Like reference numerals refer to corresponding parts throughout the several views of the drawings.

FIGS. 1A, 1B, 1C, and 1D collectively illustrate an example block diagram of a computing device in accordance with some embodiments of the present disclosure.

FIGS. 2A, 2B, 2C, and 2D collectively illustrate an example method in accordance with an embodiment of the present disclosure, in which optional steps are indicated by broken lines.

FIG. 3 illustrates an example schematic of a capture spot corresponding to a plurality of cell types for a respective plurality of cells, in accordance with some embodiments of the present disclosure.

FIG. 4 illustrates example input information, in accordance with some embodiments of the present disclosure.

FIG. 5 illustrates example output information including, for a respective current iteration of a plurality of proposed cell types, for each respective proposed cell type in the respective current iteration, for each respective capture spot in a set of capture spots, a respective proportion of cells in the respective capture spot having the respective proposed cell type, in accordance with some embodiments of the present disclosure.

FIG. 6 illustrates example output information including, for a respective current iteration of a plurality of proposed cell types, for each proposed cell type in the respective current iteration, a corresponding plurality of analytes in the proposed cell type and, for each analyte in the corresponding plurality of analytes, an abundance of the analyte, in accordance with some embodiments of the present disclosure.

FIGS. 7A, 7B, 7C, 7D, 7E, 7F, and 7G are example images including, for a respective current iteration of a plurality of proposed cell types, for each capture spot in a set of capture spots, an overlay indicating a respective proportion of cells in the respective capture spot having a proposed cell type in the current iteration, in accordance with an embodiment of the present disclosure.

FIGS. 8A and 8B are example images of a tissue sample overlaid with indications of a plurality of proposed cell types, in accordance with an embodiment of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions to determine cell type in a tissue sample.

In particular, the present disclosure provides a method for determining cell type that includes obtaining, in electronic form, input information for a set of capture spots. For each capture spot, the input information includes a corresponding position in an image of a tissue sample, and a respective abundance of each analyte in a plurality of analytes measured from the tissue sample for the capture spot. A current iteration of a plurality of proposed cell types that is set to a maximum number of proposed cell types is determined, using the respective abundance of each analyte in the plurality of analytes measured from the tissue sample for each capture spot. Each respective cell type in the current iteration of the plurality of proposed cell types has an abundance value for each analyte in the plurality of analytes. When the current iteration of the plurality of proposed cell types exceeds a minimum number of proposed cell types, a procedure is performed: a distance metric is determined between each proposed cell type in the current iteration based on the abundance value for each analyte in the plurality of analytes for each proposed cell type, and the current iteration of the plurality of proposed cell types is reformed by merging a first and a second proposed cell type having a smallest distance metric among all unique pairs of proposed cell types in the current iteration. The procedure is repeated until the current iteration of the plurality of proposed cell types matches the minimum number of proposed cell types. Output information is determined, for each respective current iteration, for each respective proposed cell type in the respective current iteration of the plurality of proposed cell types, for each respective capture spot in the set of capture spots, providing a respective proportion of cells in the respective capture spot having the respective proposed cell type.

As described above, current commercially available techniques for spatial analyte capture typically suffer from a lack of single-cell resolution, while single-cell sequencing often lacks spatial context. A topic of computational research recently under development is deconvolution of spatial analyte capture spots. For instances where each spot contains more than one cell, it can be useful to estimate the proportion of a given cell type at each spot, e.g., the mixtures of cells within the tissue microenvironment. This gives insight into the spatial organization of cells as well as how different cell types might interact in space.

Conventional methods to solve this problem use deconvolution, an approach in which typically a cell type-annotated single-cell reference is used to estimate the proportion of each reference cell category in each spot. These methods are impeded by the difficulty, and in some cases infeasibility, of obtaining relevant single-cell references. Other methods utilize a reference-free approach in which a user defines the number of cell types (e.g., a value for k) that is estimated to be in the mixture and performs deconvolution using methods generally employed in natural language processing. However, such methods typically allow for the selection of only a single number (e.g., a single value of k) prior to performing deconvolution, with no optimization. Thus, improved tools are needed in the art to adequately address such limitations.

Advantageously, the present disclosure provides solutions to the above-identified technical problem of determining cell type in a tissue sample by providing a reference-free approach that further employs a telescoping approach, allowing the user to optimize and refine the results of the deconvolution. The systems and methods disclosed herein thus have a practical application of improving the identification of cell type, e.g., within a spatial context, for the elucidation of cellular roles in tumor biology and cell interactions in the tissue microenvironment. These advantages are highlighted in Example 1 below, which illustrates that a cell type determination method in accordance with the present disclosure can achieve accuracy of cellular subclass identification comparable to that obtained using conventional reference-guided analyses and pathologist annotations.

The present disclosure further provides improvements to a technology or technical field by improving spatial analyte characterization, including but not limited to classification of cell type in biological samples, determining cell interactions within tissues, and capture spot deconvolution. In some embodiments, as further highlighted in Example 1 below, improvements to the technology or technical field include more refined deconvolution methods that advantageously allow for classification of cellular subtypes that could not be previously determined in implementations involving single-cell data or where single-cell references are not available. In this way, the presently disclosed systems and methods overcome the problems in the art, as discussed above, to allow for deconvolution of spatial data in both single-cell and reference-free contexts. Thus, the presently disclosed systems and methods not only improve the resolution of spatial data analysis, but they also advantageously facilitate the determination of new outcomes that could not previously be generated from such input data (e.g., single-cell and/or reference-free data).

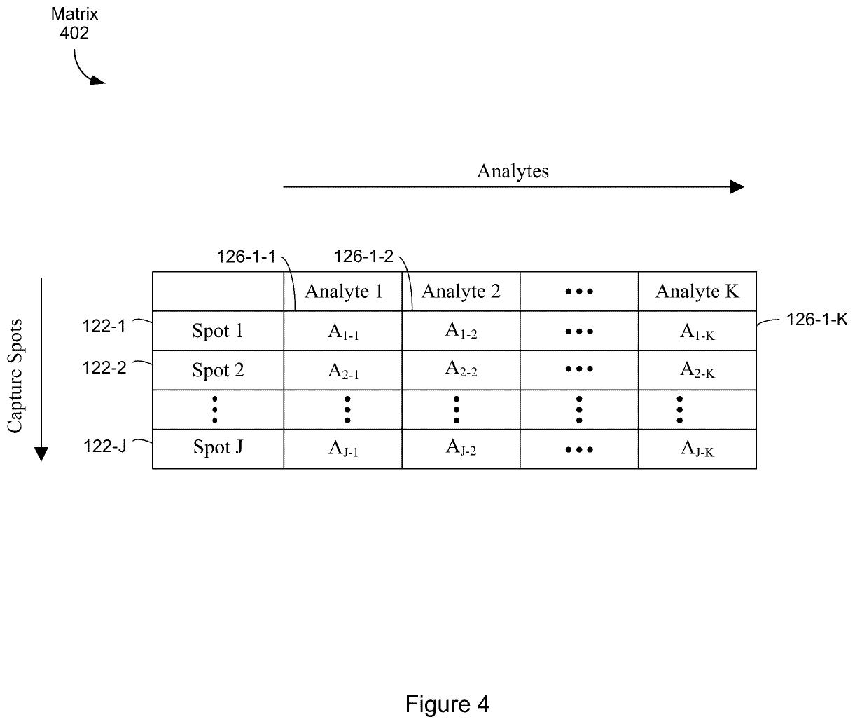

To accomplish such outcomes, in some embodiments, the presently disclosed systems and methods utilize a specific data structure that presents analyte abundances across spatially arrayed capture spots as elements (e.g., terms and/or objects) within a textual context. Optionally, this specific data structure is generated by evaluating analytes, or proportions thereof, across each capture spot in a plurality of capture spots (e.g., within a spatial context), using, for instance, text mining or natural language processing. In some embodiments, as further illustrated in Example 1, the data structure represents patterns of similarity or dissimilarity in the analyte data, optionally comprising vectors that represent proportional representations of cell types in each capture spot, and/or determined by a generative statistical model. Specific data structures, as well as corresponding output data structures, in accordance with the present disclosure are illustrated, for example, in FIGS. 4-6, and further described elsewhere herein (see, for example, the sections entitled “Determining a current iteration” and “Determining output information,” below).

Additionally, by providing a streamlined method that allows for iterative evaluation of multiple parameters (e.g., telescoping values of k), the presently disclosed systems and methods allow for faster implementation of multiple parameterized deconvolution processes compared to conventional approaches. This is due to the reduced need for repeated user-implemented selection and optimization of parameters, with subsequent initialization of each individual deconvolution after each selection process. Moreover, in some embodiments, the present disclosure provides that the iterative evaluation is automated (e.g., via telescoping and/or collapsing k) resulting in significantly faster and more efficient deconvolution due to the reduced need for separate parameter selection and deconvolution processes.

In some implementations, the present disclosure provides specific data structures that allow for increased speed and efficiency when performing spatial analysis (e.g., deconvolution and/or classification) of cell types or biological analytes. As described below, the specific data structures disclosed herein, in some embodiments, are obtained using text mining and/or natural language processing. Such approaches refer to a form of data mining that is typically used to analyze unstructured text data and advantageously allows for more rapid and efficient detection of patterns or groups in large data files, such as in large amounts of analyte and capture spot data. See, for example, the section entitled “Determining a current iteration,” below. Due to the rapid increases in size and complexity of biological datasets (e.g., sequencing and/or transcriptomics data), the present disclosure therefore allows for faster and more efficient detection of analyte abundance and spatial patterns in large analyte datasets, which in turn are used to generate the specific data structures that enhance the speed and efficiency of spatial analyte analysis.

Similarly, in some implementations, the data structures are obtained using a generative statistical model, such as a latent Dirichlet allocation model, that is further refined (e.g., via variational expectation-maximization and/or Markov chain Monte Carlo). As disclosed elsewhere herein, in some implementations, the generation of the matrix comprises, for each object in a plurality of objects (e.g., capture spots), determining term distributions (e.g., analytes) for each topic in a plurality of topics (e.g., proposed cell types). The determination of such distributions comprises, in some implementations, an expectation maximization approach. However, determining probabilities using expectation maximization is often intractable; in other words, they cannot be computationally solved in a reasonable time (e.g., within polynomial time) and/or require such extensive computational resources to complete as to be impractical or even infeasible (see, for example, the section entitled “Determining a current iteration,” below). Accordingly, the present disclosure advantageously increases the speed and efficiency of generating data structures that represent patterns of analyte abundance in spatial analyte data, while reducing the amount of computational resources required, thus improving the technological process of spatial analyte characterization.

Details of implementations are now described in conjunction with the Figures.

### General Terminology.

Specific terminology is used throughout this disclosure to explain various aspects of the apparatus, systems, methods, and compositions that are described. This sub-section includes explanations of certain terms that appear in later sections of the disclosure. To the extent that the descriptions in this section are in apparent conflict with usage in other sections of this disclosure, the definitions in this section will control.

### (i) Subject

A “subject” is an animal, such as a mammal (e.g., human or a non-human simian), or avian (e.g., bird), or other organism, such as a plant. Examples of subjects include, but are not limited to, a mammal such as a rodent, mouse, rat, rabbit, guinea pig, ungulate, horse, sheep, pig, goat, cow, cat, dog, primate (e.g., human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum.

### (ii) Nucleic Acid and Nucleotide.

The terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence-specific fashion or are capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Barcode.

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. More details on barcodes and UMIs is disclosed in United States Patent Publication No. US-2021-0155982-A1, entitled “Pipeline for Spatial Analysis of Analytes,” published May 27, 2021, and International Patent Publication No. WO2021/102039, entitled “Pipeline for Spatial Analysis of Analytes,” published May 27, 2021, each of which is hereby incorporated by reference.

### (iv) Biological Samples.

As used herein, a “biological sample” is obtained from the subject for analysis using any of a variety of techniques including, but not limited to, biopsy, surgery, and laser capture microscopy (LCM), and generally includes tissues or organs and/or other biological material from the subject. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

### System.

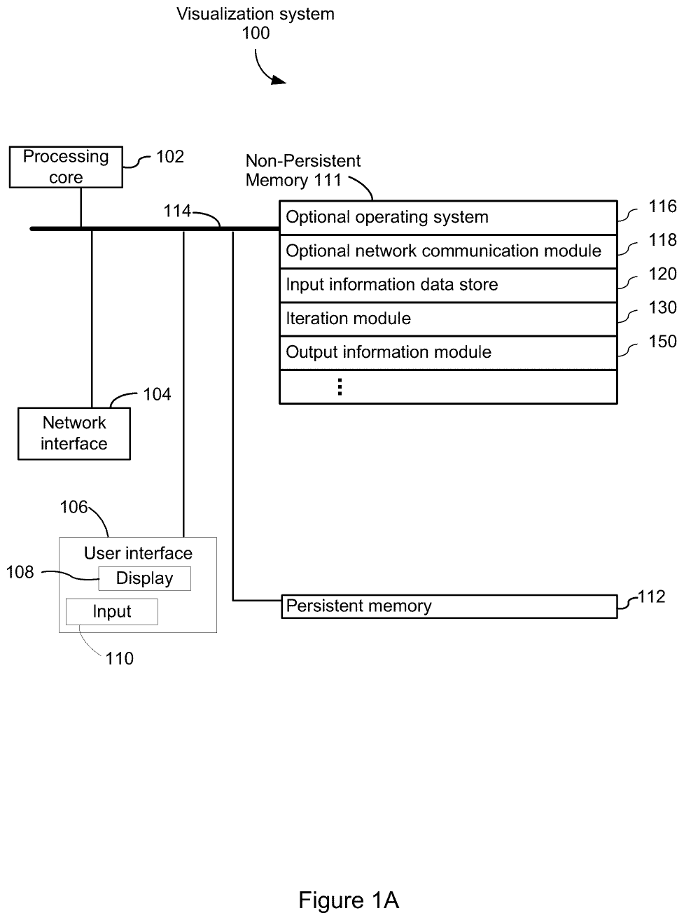

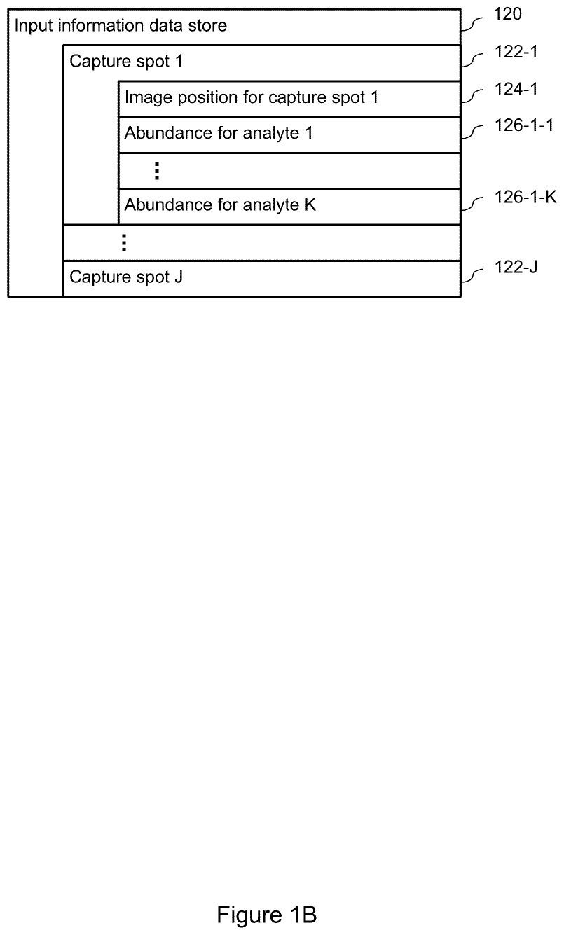

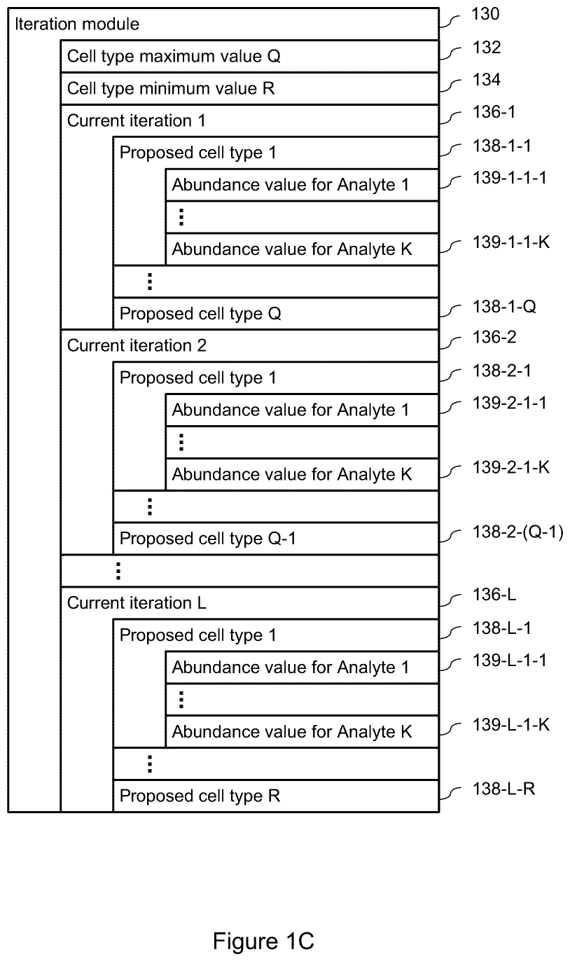

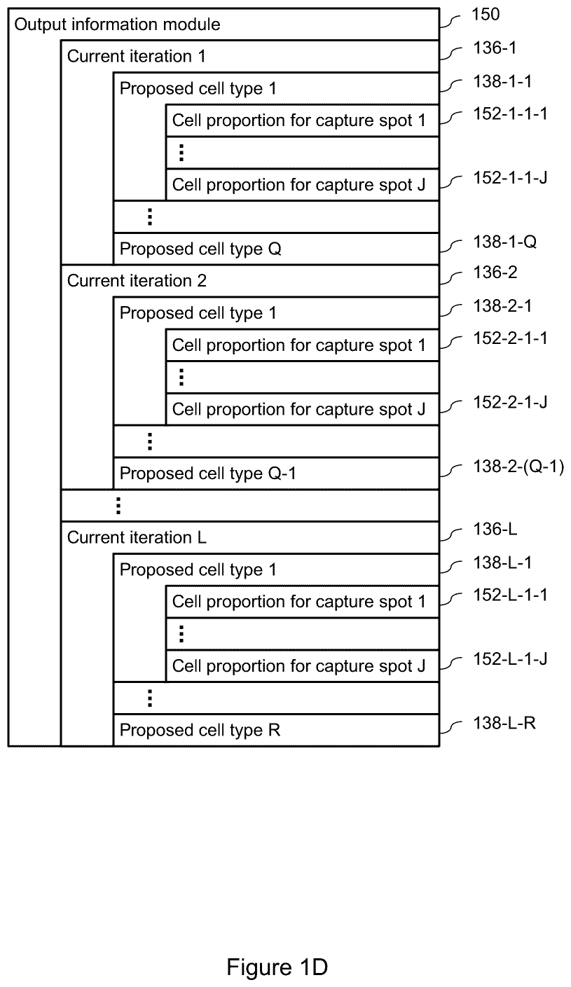

FIGS. 1A-D is a block diagram illustrating a visualization system 100 in accordance with some embodiments. The device 100 in some embodiments includes one or more processing units CPU(s) 102 (also referred to as processors), one or more network interfaces 104, a user interface 106 comprising a display 108 and an input module 110, a non-persistent 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random-access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some embodiments, the device 100 is or comprises an electronic device.

In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the visualization system 100 with other devices, or a communication network;
an input information data store 120 that optionally includes input information for a set of capture spots 122, the input information including, for each capture spot 122 (e.g., 122-1, . . . 122-J) in the set of capture spots:
   a corresponding position 124 (e.g., 124-1) in an image of a tissue sample from a subject, and
   a respective abundance 126 (e.g., 126-1-1, . . . 126-1-K) of each analyte in a plurality of analytes measured for each capture spot in the set of capture spots from the tissue sample;
an iteration module 130 that optionally:
   determines a current iteration 136 (e.g., 136-1, 136-2, . . . 136-L) of a plurality of proposed cell types 138 (e.g., 138-1-1, . . . 138-1-Q, 138-2-1, . . . 138-2-(Q−1), 138-L-1, . . . 138-L-R) that is set to a maximum number 132 of proposed cell types using the respective abundance of each analyte 126 in the plurality of analytes measured for each capture spot in the set of capture spots from the tissue sample, where each respective cell type 138 in the current iteration of the plurality of proposed cell types has an abundance value 139 (e.g., 139-1-1-1, . . . 139-1-1-K, 139-2-1-1, . . . 139-2-1-K, 139-L-1-1, . . . 139-L-1-K) for each analyte in the plurality of analytes,
   when the current iteration 136 of the plurality of proposed cell types 138 exceeds a minimum number 134 of proposed cell types, performs a procedure including determining a respective distance metric between each proposed cell type 138 in the current iteration 136 of the plurality of proposed cell types based on the abundance value 139 for each analyte in the plurality of analytes for each proposed cell type in the current iteration of the plurality of proposed cell types, and reforming the current iteration 136 of the plurality of proposed cell types 138 by merging a first proposed cell type and a second proposed cell type having a smallest distance metric among all unique pairs of proposed cell types in the current iteration of the plurality of proposed cell types, and
   repeats the procedure until the current iteration of the plurality of proposed cell types matches the minimum number 134 of proposed cell types; and
an output information module 150 that optionally determines output information, for each respective current iteration 136 of the plurality of proposed cell types, for each respective proposed cell type 138 in the respective current iteration of the plurality of proposed cell types, for each respective capture spot 122 in the set of capture spots, providing a respective proportion of cells 152 (e.g., 152-1-1-1, . . . 152-1-1-J, 152-2-1-1, . . . 152-2-1-J, 152-L-1-1, . . . 152-L-1-J) in the respective capture spot having the respective proposed cell type.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIGS. 1A-D depict a “visualization system 100,” the figures are intended more as functional description of the various features which may be present in computer systems than as a structural schematic of the implementations described herein. In practice, and as recognized by those of ordinary skill in the art, items shown separately could be combined and some items could be separated. Moreover, although FIG. 1A depicts certain data and modules in non-persistent memory 111, some or all of these data and modules may be in persistent memory 112. Further, while input information data store 120, iteration module 130, and output information module 150 are depicted as resident in non-persistent memory 111, in some embodiments, all or a portion of one or more of input information data store 120, iteration module 130, and output information module 150 is resident in persistent memory 112 at various stages of the disclosed methods.

### Methods.

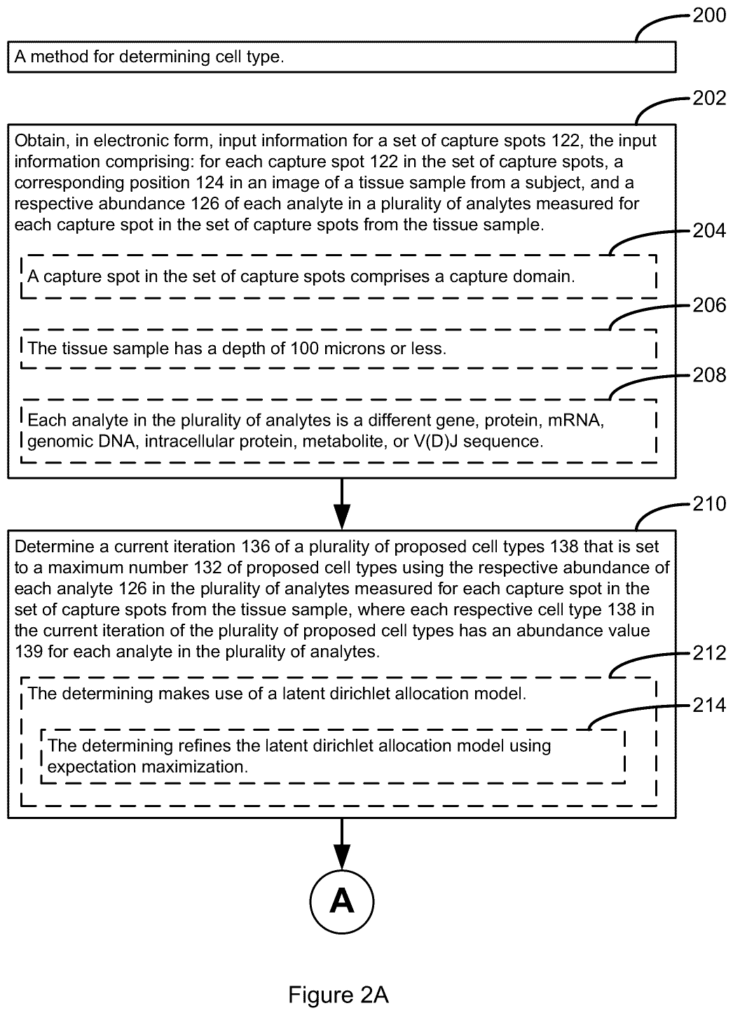

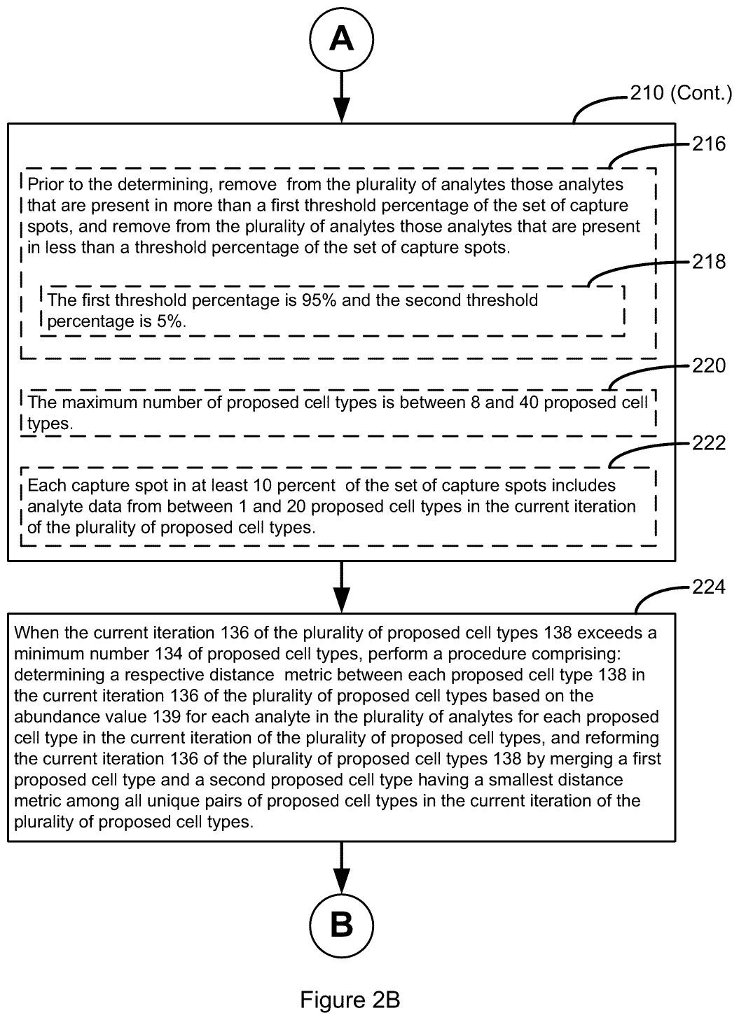

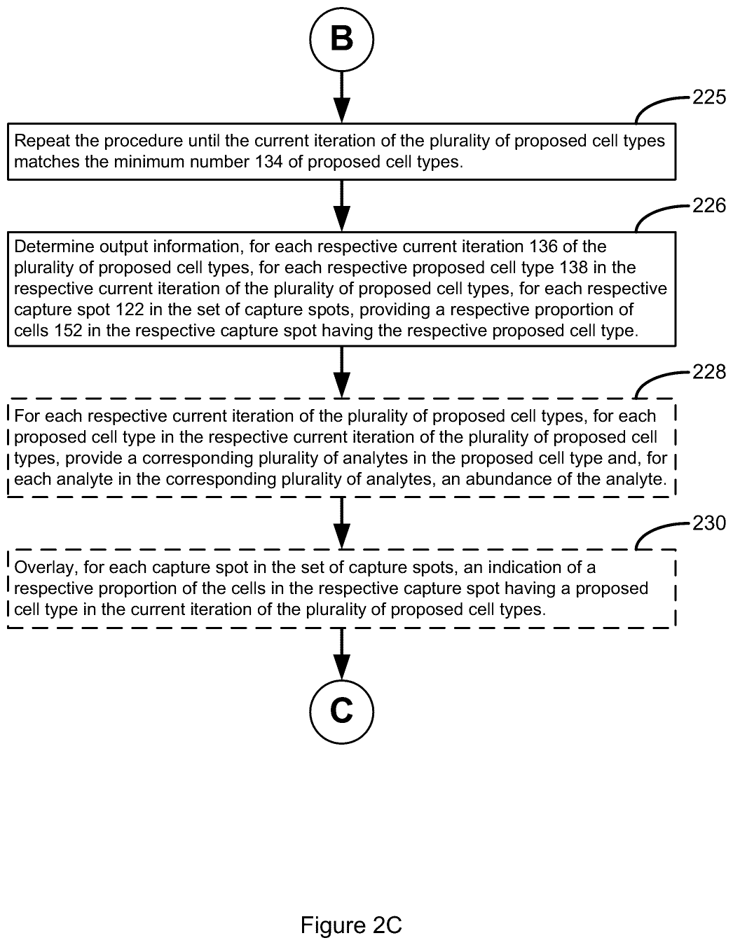

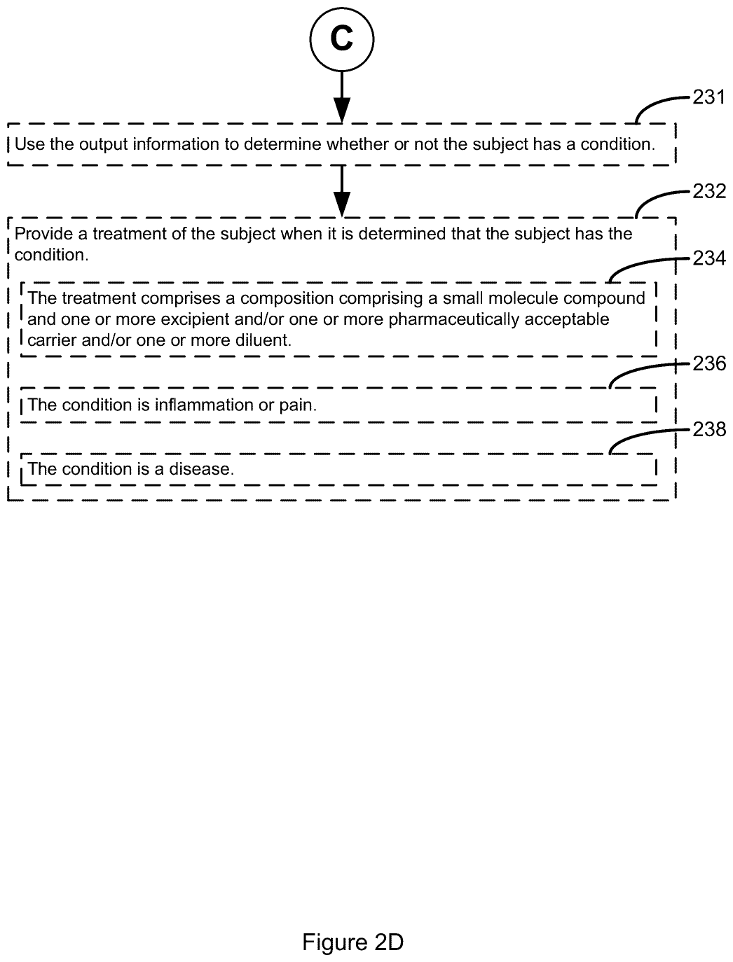

While a system in accordance with the present disclosure has been disclosed with reference to FIGS. 1A-D, a method in accordance with the present disclosure is now detailed with reference to FIGS. 2A-D.

FIGS. 2A-D collectively illustrate a method 200 for determining cell type. In some embodiments, the method is performed at a computer system comprising one or more processing cores and a memory.

### Block 202—Obtaining Information.

Referring to block 202, the method includes obtaining, in electronic form, input information for a set of capture spots 122. The input information includes, for each capture spot 122 in the set of capture spots, a corresponding position 124 in an image of a tissue sample from a subject, and a respective abundance 126 of each analyte in a plurality of analytes measured for each capture spot in the set of capture spots from the tissue sample.

In some embodiments, each respective capture spot in the set of capture spots encompasses (e.g., includes analyte abundances from) at least 1, at least 2, at least 3, at least 4, at least 5, at least 6, at least 7, at least 8, at least 9, at least 10 cells, at least 20, or at least 30 cells in the tissue sample. In some embodiments, each respective capture spot in the set of capture spots encompasses (e.g., includes analyte abundances from) no more than 50, no more than 30, no more than 20, no more than 10, or no more than 5 cells in the tissue sample. In some embodiments, each respective capture spot in the set of capture spots encompasses (e.g., includes analyte abundances obtained) from 1 to 10, from 1 to 5, from 2 to 4, from 1 to 30, from 3 to 20, or from 10 to 50 cells in the tissue sample. In some embodiments, each respective capture spot in the set of capture spots encompasses (e.g., includes analyte abundances from) another range of cells starting no lower than 1 cell and ending no higher than 50 cells.

In some embodiments, the number of cells encompassed by each respective capture spot is determined based on the tissue sample and/or the cell density thereof. In some embodiments, a first respective capture spot in the set of capture spots encompasses a different number of cells in the tissue sample as a second respective capture spot in the set of capture spots. In some embodiments, each respective capture spot in the set of capture spots encompasses the same number of cells in the tissue sample.

In some embodiments, the tissue sample includes a plurality of cell types (e.g., a mixture of cell types). Alternatively or additionally, in some embodiments, the tissue sample includes a plurality of cells, where at least a first cell in the plurality of cells is of a different cell type than at least a second cell in the plurality of cells.

In some embodiments, the plurality of cell types includes at least 1, at least 2, at least 3, at least 4, at least 5, at least 6, at least 7, at least 8, at least 9, at least 10, at least 20, at least 30, at least 40, at least 50, at least 100, or at least 200 cell types. In some embodiments, the plurality of cell types includes no more than 500, no more than 200, no more than 100, no more than 50, no more than 30, no more than 20, no more than 10, or no more than 5 cell types. In some embodiments, the plurality of cell types includes from 1 to 10, from 1 to 5, from 2 to 4, from 1 to 30, from 3 to 20, from 10 to 50, from 40 to 200, or from 100 to 500 cell types. In some embodiments, the plurality of cell types falls within another range of cells starting no lower than 1 cell type and ending no higher than 500 cell types.

In some embodiments, each respective capture spot in the set of capture spots includes (e.g., encompasses a plurality of cells collectively representing) a plurality of cell types. In some embodiments, each respective capture spot in the set of capture spots includes at least 1, at least 2, at least 3, at least 4, at least 5, at least 6, at least 7, at least 8, at least 9, at least 10, at least 20, or at least 30 cell types. In some embodiments, each respective capture spot in the set of capture spots includes no more than 50, no more than 30, no more than 20, no more than 10, or no more than 5 cell types. In some embodiments, each respective capture spot in the set of capture spots includes from 1 to 10, from 1 to 5, from 2 to 4, from 1 to 30, from 3 to 20, or from 10 to 50 cell types. In some embodiments, each respective capture spot in the set of capture spots falls within another range of cell types starting no lower than 1 cell type and ending no higher than 50 cell types.

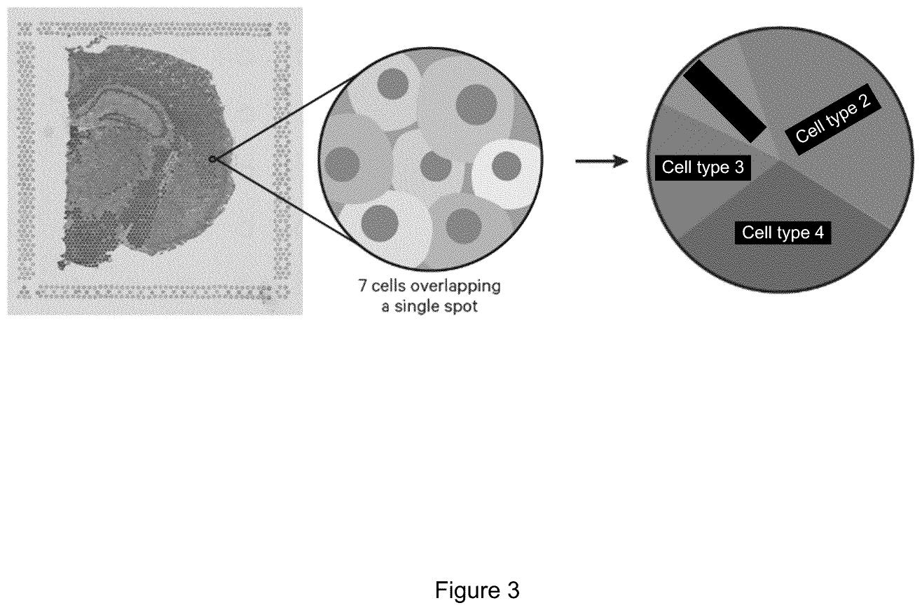

For instance, FIG. 3 provides an example schematic of a capture spot that encompasses a subset of cells in a plurality of cells in the tissue sample (e.g., 7 cells overlapping a single capture spot). The subset of cells encompassed by the capture spot collectively represents a plurality of cell types, such that the capture spot is characterized by various corresponding proportions of cell types (e.g., Cell type 1, Cell type 2, Cell type 3, and Cell type 4).

In some embodiments, at least a first respective capture spot in the set of capture spots has a different proportion of cell types than at least a second respective capture spot in the set of capture spots. In some embodiments, each respective capture spot in the set of capture spots has the same proportion of cell types in the plurality of cell types.

In some embodiments, the plurality of cell types includes one or more disease states, tissue types, organ types, species, assay conditions and/or any other feature or factor that allows for the differentiation of cells (or groups of cells) from one another. In some embodiments, the plurality of cell types includes healthy and/or diseased cells. In some embodiments, the plurality of cell types includes differentiated cells, such as immune cells. In some embodiments, the plurality of cell types includes cancer cells. Alternatively or additionally, in some embodiments, the plurality of cell types consists of human cell types. Non-limiting examples of cell types suitable for use in the present disclosure include invasive carcinoma, ductal carcinoma in situ, immune, stromal compartments, myoepithelial, macrophages, invasive tumor, and/or dendritic cells. Various cell types are known in the art and are contemplated for use herein, as described, for instance, in Sender et al., “Revised estimates for the number of human and bacteria cells in the body,” PLOS Biol. 2016; 14(8): e1002533; and Regev et al., “The human cell atlas,” Elife. 2017; 6:e27041, each of which is hereby incorporated herein by reference in its entirety.

Referring to block 204, in some embodiments, a capture spot in the set of capture spots includes a capture domain.

In some embodiments, a capture spot in the set of capture spots comprises a cleavage domain.

In some embodiments, each capture spot in the set of spots is attached directly or attached indirectly to a substrate.

In some embodiments, each capture spot in the set of spots is not attached to a substrate. For instance, in some embodiments, microfluidic partitions are used to partition very small numbers of analytes and to barcode those partitions. In some such embodiments, where analyte abundances are measured from single cells, the microfluidic partitions are used to capture individual cells within each microfluidic droplet and then pools of single barcodes within each of those droplets are used to tag all the contents of a given cell.

In some embodiments, the information for the set of capture spots is obtained from the tissue sample mounted on a substrate.

Non-limiting examples of suitable methods and embodiments for obtaining information (e.g., analyte abundances and/or images) from tissue samples, including sample preparation, library preparation, capture spots, microfluidic partitions, substrates, and/or sequencing, contemplated for use in the present disclosure are described in further detail in United States Patent Publication No. US-2021-0155982-A1, entitled “Pipeline for Spatial Analysis of Analytes,” published May 27, 2021; International Patent Publication No. WO2021/102039, entitled “Pipeline for Spatial Analysis of Analytes,” published May 27, 2021; and U.S. Pat. No. 11,514,575B2, entitled “Systems and methods for identifying morphological patterns in tissue samples,” published Nov. 29, 2022, each of which is hereby incorporated by reference.

In some embodiments, each capture spot in the set of capture spots has a unique spatial barcode that encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10^{12}.}

In some embodiments, the set of capture spots comprises 1000 capture spots. In some embodiments, the set of capture spots comprises 2000, 3000, 4000, 5000, 6000, 7000, 8000, 9000, or 10000 capture spots.

In some embodiments, the set of capture spots comprises at least 50, at least 100, at least 200, at least 300, at least 400, at least 500, at least 1000, at least 2000, at least 3000, at least 4000, at least 5000, at least 10,000, at least 15,000, at least 20,000, at least 40,000, at least 100,000, at least 500,000, or at least 1 million capture spots. In some embodiments, the set of capture spots comprises no more than 10 million, no more than 1 million, no more than 500,000, no more than 100,000, no more than 50,000, no more than 20,000, no more than 10,000, no more than 5000, no more than 1000, no more than 500, or no more than 100 capture spots. In some embodiments, the set of capture spots comprises from 100 to 500, between 500 and 1000, from 1000 to 5000, from 5000 to 10,000, from 10,000 to 15,000, from 15,000 to 20,000, from 10,000 to 50,000, from 30,000 to 100,000, from 100,000 to 500,000, or from 500,000 to 10 million capture spots. In some embodiments, the set of capture spots falls within another range starting no lower than 50 capture spots and ending no higher than 10 million capture spots.

In some embodiments, each respective capture spot in the set of capture spots includes a plurality of capture probes. In some embodiments, each respective capture spot in the set of capture spots includes 1000 or more capture probes, 2000 or more capture probes, 10,000 or more capture probes, 100,000 or more capture probes, 1×10⁶ or more capture probes, 2×10⁶ or more capture probes, or 5×10⁶ or more capture probes. In some embodiments, the plurality of capture probes includes no more than 1×10⁷, no more than 1×10⁶, no more than 100,000, no more than 50,000, no more than 10,000, no more than 5000, no more than 2000, or no more than 1000 capture probes. In some embodiments, the plurality of capture probes is from 500 to 10,000, from 5000 to 100,000, from 1000 to 1×10⁶, from 10,000 to 500,000, or from 1×10⁶ to 1×10⁷ capture probes. In some embodiments, the plurality of capture probes falls within another range starting no lower than 500 capture probes and ending no higher than 1×10⁷ capture probes.

In some embodiments, each capture probe in the respective capture spot includes a poly-A sequence or a poly-T sequence and the unique spatial barcode that characterizes the respective capture spot.

In some embodiments, each capture probe in the respective capture spot includes the same spatial barcode from a plurality of spatial barcodes. In some embodiments, each capture probe in the respective capture spot includes a different spatial barcode from a plurality of spatial barcodes. In some embodiments, the plurality of spatial barcodes includes at least 50, at least 100, at least 1000, at least 10,000, at least 100,000, at least 1×10⁶, at least 1×10⁷, at least 1×10⁸, at least 1×10⁹, at least 1×10¹⁰, at least 1×10¹¹, or at least 1×10¹² barcodes. In some embodiments, the plurality of barcodes includes no more than 1×10¹³, no more than 1×10¹², no more than 1×10¹¹, no more than 1×10¹⁰, no more than 1×10⁹, no more than 1×10⁸, no more than 1×10⁷, no more than 1×10⁶, no more than 100,000, no more than 10,000, no more than 1000, or no more than 100 barcodes. In some embodiments, the plurality of barcodes consists of from 50 to 10,000, from 1000 to 1×10⁶, from 100,000 to 1×10⁸, from 1×10⁶ to 1×10⁹, from 1×10⁸ to 1×10¹¹, or from 1×10¹⁰ to 1×10¹³ barcodes. In some embodiments, the plurality of spatial barcodes falls within another range starting no lower than 50 spatial barcodes and ending no higher than 1×10¹³ spatial barcodes. In some embodiments, a spatial barcode is selected from a set of barcodes, where the set of barcodes is represented as a set selected from the group consisting of: {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

In some embodiments, a respective capture spot in the set of capture spots includes a respective plurality of capture probes, where each capture probe in the plurality of capture probes includes a capture domain that is characterized by a capture domain type in a plurality of capture domain types, and each respective capture domain type in the plurality of capture domain types is configured to bind to a different analyte in the plurality of analytes.

In some embodiments, the plurality of capture domain types comprises between 5 and 15,000 capture domain types and the respective plurality of capture probes (e.g., capture probe plurality) includes at least five, at least 10, at least 100, or at least 1000 capture probes for each capture domain type in the plurality of capture domain types. In some embodiments, the respective capture probe plurality includes no more than 5000, no more than 1000, no more than 100, or no more than 10 capture probes for each capture domain type in the plurality of capture domain types. In some embodiments, the respective capture probe plurality includes from 5 to 100, from 10 to 500, from 100 to 1000, or from 500 to 5000 capture probes for each capture domain type in the plurality of capture domain types. In some embodiments, the respective capture probe plurality falls within another range starting no lower than 5 capture probes and ending no higher than 5000 capture probes.

In some embodiments, a respective capture spot in the set of capture spots includes a plurality of capture probes, where each capture probe in the plurality of capture probes includes a capture domain that is characterized by a single capture domain type configured to bind to each analyte in the plurality of analytes in an unbiased manner.

In some embodiments, each respective capture spot in the set of capture spots is contained within a 100 micron by 100 micron square on the substrate.

In some embodiments, a distance between a center of each respective capture spot to a neighboring capture spot in the set of capture spots on the substrate is between 10 microns and 100 microns. In some embodiments, a shape of each capture spot in the set of capture spots on the substrate is a closed-form shape. In some embodiments, the closed-form shape is circular, elliptical, or an N-gon, where N is a value between 1 and 20. In some embodiments, the closed-form shape is circular and each capture spot in the set of capture spots has a diameter of 80 microns or less. In some embodiments, the closed-form shape is circular and each capture spot in the set of capture spots has a diameter of between 30 microns and 65 microns. In some embodiments, a distance between a center of each respective capture spot to a neighboring capture spot in the set of capture spots on the substrate is between 50 microns and 80 microns.

Non-limiting examples of suitable capture spots contemplated for use in the present disclosure are described in further detail in United States Patent Publication No. US-2021-0155982-A1, entitled “Pipeline for Spatial Analysis of Analytes,” published May 27, 2021; International Patent Publication No. WO2021/102039, entitled “Pipeline for Spatial Analysis of Analytes,” published May 27, 2021; and U.S. Pat. No. 11,514,575B2, entitled “Systems and methods for identifying morphological patterns in tissue samples,” published Nov. 29, 2022, each of which is hereby incorporated by reference.

Referring to block 206, in some embodiments, the tissue sample has a depth of 100 microns or less.

In some embodiments, the tissue sample is a tissue section (e.g., a sectioned tissue sample). In some embodiments, the tissue sample has a depth of 500 microns or less, 100 microns or less, 80 microns or less, 70 microns or less, 60 microns or less, 50 microns or less, 40 microns or less, 25 microns or less, 20 microns or less, 15 microns or less, 10 microns or less, 5 microns or less, 2 microns or less, or 1 micron or less. In some embodiments, the tissue sample has a depth of at least 0.1 microns, at least 1 micron, at least 5 microns, at least 10 microns, at least 15 microns, at least 20 microns, at least 30 microns, at least 50 microns, or at least 80 microns. In some embodiments, the tissue sample has a depth of between 10 microns and 20 microns, between 1 and 10 microns, between 0.1 and 5 microns, between 20 and 100 microns, between 1 and 50 microns, or between 0.5 and 10 microns. In some embodiments, the tissue sample falls within another range starting no lower than 0.1 microns and ending no higher than 500 microns.

In some embodiments, the tissue sample comprises a plurality of cells. In some embodiments, the plurality of cells includes 500 or more cells, 5000 or more cells, 100,000 or more cells, 250,000 or more cells, 500,000 or more cells, 1,000,000 or more cells, 10 million or more cells or 50 million or more cells. In some embodiments, the plurality of cells includes no more than 100 million, no more than 50 million, no more than 10 million, no more than 1 million, no more than 500,000, no more than 250,000, no more than 100,000, or no more than 5000 cells. In some embodiments, the plurality of cells includes from 500 to 10,000, from 1000 to 100,000, from 50,000 to 500,000, from 100,000 to 1 million, or from 1 million to 100 million cells. In some embodiments, the plurality of cells falls within another range starting no lower than 500 cells and ending no higher than 100 million cells.

In some embodiments, the tissue sample consists of a plurality of dissociated cells. In some embodiments, the tissue sample is not dissociated.

Further embodiments of biological samples, including tissue samples, are provided herein (see, “General Terminology: (iv) Biological Samples,” above).

In some embodiments, the plurality of analytes comprises DNA, RNA, proteins, or a combination thereof. For instance, in some embodiments, each respective analyte in the plurality of analytes is the same type of analyte. In some embodiments, the plurality of analytes includes at least an analyte of a first type (e.g., RNA molecule) and an analyte of a second type (e.g., protein). In some embodiments, the plurality of analytes comprises a plurality of analyte types (e.g., RNA and protein, RNA and DNA, DNA and protein, or a combination of RNA, DNA, and protein).

Referring to block 208, in some embodiments, each analyte in the plurality of analytes is a different gene, protein, mRNA, genomic DNA, intracellular protein, metabolite, or V(D)J sequence.

In some embodiments, a respective abundance of a respective analyte is a count of molecules for the respective analyte that was measured in the tissue sample at the corresponding capture spot. For instance, in some embodiments, each respective abundance is a count of transcript reads within the cell that map to a respective gene in a plurality of genes. In some embodiments, the input information includes a plurality of abundances for the plurality of analytes across the set of capture spots, where the plurality of abundances represents a whole transcriptome experiment that quantifies gene expression from the tissue sample in counts of transcript reads mapped to the genes. Example input information, including a respective abundance for a respective analyte in the plurality of analytes measured for each capture spot in the set of capture spots from the tissue sample, is illustrated, for instance, in FIGS. 1B and 4.

In some embodiments, a respective abundance of a respective analyte is a relative abundance of the respective analyte that was measured in the tissue sample at the corresponding capture spot, relative to one or more analytes other than the respective analyte. For instance, in some embodiments, the respective abundance of a respective analyte is a relative abundance of the respective analyte normalized against one or more housekeeping analytes (e.g., housekeeping genes) or reference analytes. In some embodiments, the respective abundance of a respective analyte is an abundance of the respective analyte in a first capture spot in the set of capture spots normalized against the abundance of the respective analyte in one or more other capture spots, other than the first capture spot, in the set of capture spots. In some embodiments, the respective abundance of a respective analyte is a differential value (e.g., differential expression), as described below. In some embodiments, the respective abundance of a respective analyte is an abundance of the respective analyte in a first cluster of capture spots in a plurality of capture spot clusters normalized against the abundance of the respective analyte in one or more other capture spot clusters, other than the first cluster, in the plurality of clusters, as described below.

Non-limiting examples of suitable methods and embodiments for obtaining and pre-processing analyte abundances, including obtaining counts, normalization, clustering, and/or determining differential values, contemplated for use in the present disclosure are described in further detail in United States Patent Publication No. US-2021-0155982-A1, entitled “Pipeline for Spatial Analysis of Analytes,” published May 27, 2021; International Patent Publication No. WO2021/102039, entitled “Pipeline for Spatial Analysis of Analytes,” published May 27, 2021; and U.S. Pat. No. 11,514,575B2, entitled “Systems and methods for identifying morphological patterns in tissue samples,” published Nov. 29, 2022, each of which is hereby incorporated by reference.

In some embodiments, the plurality of analytes comprises five or more analytes, ten or more analytes, fifty or more analytes, one hundred or more analytes, five hundred or more analytes, 1000 or more analytes, 2000 or more analytes, or between 2000 and 100,000 analytes.

In some embodiments, the plurality of analytes comprises 100, 200, 300, 400, 500, or 1000 analytes.

In some embodiments, the plurality of analytes comprises at least 5, at least 10, at least 20, at least 50, at least 100, at least 200, at least 300, at least 400, at least 500, at least 600, at least 700, at least 800, at least 900, at least 1000, at least 2000, at least 3000, at least 5000, at least 6000, at least 7000, at least 8000, at least 9000, at least 10,000, at least 20,000, at least 30,000, at least 40,000, at least 50,000, at least 100,000, at least 200,000, or at least 300,000 analytes. In some embodiments, the plurality of analytes comprises no more than 500,000, no more than 200,000, no more than 100,000, no more than 80,000, no more than 50,000, no more than 30,000, no more than 20,000, no more than 10,000, no more than 5000, no more than 3000, no more than 2000, no more than 1000, no more than 500, no more than 100, or no more than 50 analytes. In some embodiments, the plurality of analytes comprises between 5 and 2000, between 1000 and 100,000, between 2000 and 10,000, between 5000 and 50,000, between 50 and 5000, or between 100 and 10,000 analytes. In some embodiments, the plurality of analytes falls within another range starting no lower than 5 analytes and ending no higher than 500,000 analytes.

Non-limiting examples of suitable biological samples and/or analytes contemplated for use in the present disclosure are described in further detail in United States Patent Publication No. US-2021-0155982-A1, entitled “Pipeline for Spatial Analysis of Analytes,” published May 27, 2021; International Patent Publication No. WO2021/102039, entitled “Pipeline for Spatial Analysis of Analytes,” published May 27, 2021; and U.S. Pat. No. 11,514,575B2, entitled “Systems and methods for identifying morphological patterns in tissue samples,” published Nov. 29, 2022, each of which is hereby incorporated by reference.

In some embodiments, the method includes clustering the plurality of analyte abundances, including a respective abundance of each analyte in a plurality of analytes measured for each capture spot in the set of capture spots from the tissue sample.

Any one of a number of clustering techniques can be used, examples of which include, but are not limited to, dimension reduction techniques.

For example, principal component analysis (PCA) is a mathematical procedure that reduces a number of correlated variables into fewer uncorrelated variables called “principal components.” The first principal component is selected such that it accounts for as much of the variability in the data as possible, and each succeeding component accounts for as much of the remaining variability as possible. The purpose of PCA is to discover or to reduce the dimensionality of the dataset, and to identify new meaningful underlying variables. PCA is accomplished by establishing actual data in a covariance matrix or a correlation matrix. The mathematical technique used in PCA is called Eigen analysis: one solves for the eigenvalues and eigenvectors of a square symmetric matrix with sums of squares and cross products. The eigenvector associated with the largest eigenvalue has the same direction as the first principal component. The eigenvector associated with the second largest eigenvalue determines the direction of the second principal component. The sum of the eigenvalues equals the trace of the square matrix, and the maximum number of eigenvectors equals the number of rows (or columns) of this matrix. See, for example, Duda, Hart, and Stork, Pattern Classification, Second Edition, John Wiley & Sons, Inc., NY, 2000, pp. 115-116, which is hereby incorporated by reference.

In some embodiments, principal component analysis, or other forms of data reduction, such as subset selection (e.g., as disclosed in Hastic, 2001, The Elements of Statistical Learning, Springer, New York, pp. 55-57), discrete methods (e.g., as disclosed in Furnival & Wilson, 1974, “Regression by Leaps and Bounds,” Technometrics 16(4), 499-511), forward/backward stepwise selection (e.g., as disclosed in Berk, 1978, “Comparing Subset Regression Procedures,” Technometrics 20:1, 1-6), shrinkage methods (e.g., as disclosed in Hastie, 2001, The Elements of Statistical Learning, Springer, New York, pp. 59-66), ridge regression (e.g., as disclosed in Hastic, 2001, The Elements of Statistical Learning, Springer, New York, pp. 59-64), lasso techniques (e.g., as disclosed in Hastie, 2001, The Elements of Statistical Learning, Springer, New York, pp. 64-65, 69-72, 330-331), derived input direction methods (e.g., principal component regression (PCR), partial least squares (PLS), etc. as disclosed, for example, in Viyayakurma and Schaal, 2000, “Locally Weighted Projection Regression: An O(n) Algorithm for Incremental Real Time Learning in High Dimensional Space, Proc. of Seventeenth International Conference on Machine Learning (ICML2000), pp. 1079-1086), or combinations thereof, are used to reduce the dimensionality of the analyte abundance data, where dimensions are termed principal components or features.

For clustering in accordance with one embodiment of the systems and methods of the present disclosure, regardless at what stage it is performed, consider the case in which each capture spot 122 is associated with ten analytes. In such instances, each capture spot can be expressed as a vector:

\vec{X}₁₀={x₁,x₂,x₃,x₄,x₅,x₆,x₇,x₈,x₉,x₁₀}

where Xᵢ is the abundance 126 for the analyte i associated with capture spot 122. Thus, if there are one thousand capture spots 122, 1000 analyte vectors are defined. Those capture spots 122 that exhibit similar analyte abundances across the plurality of analytes will tend to cluster together. For instance, in a reduced case where each capture spot is an individual cell, the analytes correspond to mRNA mapped to individual genes within such individual cells, and the abundances 126 are mRNA counts for such mRNA, it is the case in some embodiments that the input information includes mRNA data from one or more cell types, two or more cell types, three or more cell types, etc. In such instances, it is expected that cells of like type will tend to have like values for mRNA across the set of genes (mRNA) and therefore cluster together. For instance, if the input information includes class a: cells from a first cell type, and class b: cells from a second cell type, an ideal clustering model will cluster the analyte abundances 126 into two groups, with one cluster group uniquely representing class a and the other cluster group uniquely representing class b.

Clustering is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 216 of Duda 1973.

Once a method for measuring “similarity” or “dissimilarity” between points in a dataset has been selected, clustering requires a criterion function that measures the clustering quality of any partition of the data. Partitions of the dataset that extremize the criterion function are used to cluster the data. See page 217 of Duda 1973. Criterion functions are discussed in Section 6.8 of Duda 1973.

More recently, Duda et al., Pattern Classification, second edition, John Wiley & Sons, Inc. New York, which is hereby incorporated by reference, has been published. Pages 537-563 describe clustering in detail. More information on clustering techniques can be found in Kaufman and Roussecuw, 1990, Finding Groups in Data: An Introduction to Cluster Analysis, Wiley, New York, N.Y.; Everitt, 1993, Cluster analysis (Third Edition), Wiley, New York, N. Y.; and Backer, 1995, Computer-Assisted Reasoning in Cluster Analysis, Prentice Hall, Upper Saddle River, N.J. Exemplary clustering techniques that can be used in the systems and methods of the present disclosure to cluster the analyte abundances include, but are not limited to, graph-based clustering, non-graph-based clustering, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and/or Jarvis-Patrick clustering.

Non-limiting example methods for clustering analyte abundances are further described, for instance, in United States Patent Publication No. US-2021-0155982-A1, entitled “Pipeline for Spatial Analysis of Analytes,” published May 27, 2021; and International Patent Publication No. WO2021/102039, entitled “Pipeline for Spatial Analysis of Analytes,” published May 27, 2021, each of which is hereby incorporated herein by reference in its entirety.

In some embodiments, the method includes determining, for each respective analyte in the plurality of analytes for each respective cluster in a plurality of clusters, a difference in the abundance for the respective analyte across the respective subset of capture spots in the respective cluster relative to the abundance for the respective analyte across the plurality of clusters other than the respective cluster, thereby deriving a differential value (e.g., differential expression) for each respective analyte in the plurality of analytes for each cluster in the plurality of clusters.

Various non-limiting methods for obtaining differential values (e.g., expression) suitable for use in the present disclosure are described, for example, in United States Patent Publication No. US-2021-0155982-A1, entitled “Pipeline for Spatial Analysis of Analytes,” published May 27, 2021; and International Patent Publication No. WO2021/102039, entitled “Pipeline for Spatial Analysis of Analytes,” published May 27, 2021, each of which is hereby incorporated herein by reference in its entirety.

Given that measurement of abundances for analytes (e.g., count of mRNA that maps to a given gene in a given capture spot) is typically noisy, the variance of the abundances for analytes in each capture spot (e.g., count of mRNA that maps to given gene in a given capture spot) in a given cluster of such capture spots is taken into account in some embodiments. This is analogous to the t-test which is a statistical way to measure the difference between two samples. Here, in some embodiments, statistical methods that take into account that a discrete number of analytes are being measured (as the abundances 126 for a given analyte) for each capture spot 122 and that model the variance that is inherent in the system from which the measurements are made are implemented.

In some embodiments, each abundance 126 is normalized prior to computing the differential value for each respective analyte in the plurality of analytes for each respective cluster in the plurality of clusters. In some embodiments, the normalizing comprises modeling the abundance 126 of each analyte associated with each capture spot in the set of capture spots with a negative binomial distribution having a consensus estimate of dispersion, e.g., without loading the entire dataset into non-persistent memory 111. Such embodiments are useful, for example, for RNA-seq experiments that produce abundances 126 for analytes (e.g., digital counts of mRNA reads that are affected by both biological and technical variation). To distinguish the systematic changes in expression between conditions from noise, the counts are frequently modeled by the Negative Binomial distribution.

In some embodiments, the plurality of analytes has higher-than-expected expression variance across the set of capture spots.

For instance, in some embodiments, the negative binomial distribution for an abundance 126 for a given analyte includes a dispersion parameter for the abundance 126 that tracks the extent to which the variance in the abundance 126 exceeds an expected value. See Yu, 2013, “Shrinkage estimation of dispersion in Negative Binomial models for RNA-seq experiments with small sample size,” Bioinformatics 29, pp. 1275-1282, and Cameron and Trivedi, 1998, “Regression Analysis of Count Data,” Econometric Society Monograph 30, Cambridge University Press, Cambridge, UK, each of which is hereby incorporated by reference. In some embodiments, the plurality of analytes does not have higher-than-expected expression variance across the set of capture spots.

Various non-limiting methods for normalizing analyte abundances suitable for use in the present disclosure are described, for example, in United States patenttent Publication No. US-2021-0155982-A1, entitled “Pipeline for Spatial Analysis of Analytes,” published May 27, 2021; and International Patent Publication No. WO2021/102039, entitled “Pipeline for Spatial Analysis of Analytes,” published May 27, 2021, each of which is hereby incorporated herein by reference in its entirety.

### Block 210—Determining a Current Iteration.

Referring to block 210, the method further includes determining a current iteration 136 of a plurality of proposed cell types 138 that is set to a maximum number 132 of proposed cell types using the respective abundance of each analyte 126 in the plurality of analytes measured for each capture spot in the set of capture spots from the tissue sample, where each respective cell type 138 in the current iteration of the plurality of proposed cell types has an abundance value 139 for each analyte in the plurality of analytes.

In some embodiments, the determining makes use of a text mining method on the respective abundance of each analyte in the plurality of analytes measured for each capture spot in the set of capture spots from the tissue sample. Text mining refers to a form of data mining that is typically used to analyze unstructured text data. Such methods advantageously allow for the rapid and efficient detection of patterns or groups, often referred to as topics, in large data files, which are rapidly increasing in size and number due to the development of modern information systems able to capture and digitize vast amounts of information. Typically, text mining methods evaluate a natural language context of all or a portion of a text corpus (e.g., a body of text). A text corpus used for text mining includes a plurality of objects (e.g., documents, sections, paragraphs, sentences, or any portion or combination thereof) collectively comprising a plurality of terms (e.g., words). For each respective object in the plurality of objects, for each term in the plurality of terms, a corresponding frequency of the respective term in the respective object is obtained. In some embodiments of the present disclosure, the text corpus is represented by a collective plurality of abundances including the respective abundance of each analyte in the plurality of analytes measured for each capture spot in the set of capture spots from the tissue sample. In some such embodiments, terms are represented by analytes, objects are represented by capture spots, and topics are represented by cell types.

In some embodiments, the text corpus is obtained in the form of a matrix dimensioned by objects and terms. In other words, in an example embodiment, for each respective document in a plurality of documents, the text corpus includes a count of the number of times a particular word is observed in the respective document. As an example, a collection of d documents can be represented in a space of t terms by a matrix d×t where each entry is the frequency of occurrence of each term in the plurality of terms, in each document in the plurality of documents. An example illustration of such a matrix 402 is provided in FIG. 4, where objects are represented by a set of capture spots 122 (e.g., 122-1, 122-2, . . . 122-J) and terms are represented by a plurality of analytes. Entries in the example matrix 402 include the analyte abundances shown in FIG. 1B, including a respective abundance 126 (e.g., 126-1-1, 126-1-2, . . . 126-1-K) of each analyte in the plurality of analytes measured for each capture spot 122 in the set of capture spots from the tissue sample.

In some embodiments, the text mining method employs a “bag of words” approach, in which the order of terms in a respective object is not considered, but rather the frequencies of the terms regardless of order of appearance or relative position. In some embodiments, the plurality of terms is selected to include one or more target terms (e.g., terms that are of interest, informative, or associated with a particular topic). In some embodiments, the plurality of terms consists of a plurality of target terms. Alternatively or in addition, in some embodiments, the text mining method includes removing one or more terms from the plurality of terms in order to remove non-target terms (e.g., terms that are not of interest, uninformative, or not associated with a particular topic).

In some embodiments, the text mining method employs a vector space model (VSM). As indicated above, in some embodiments, the text corpus is obtained in the form of matrix dimensioned by objects and terms. Thus, in some such embodiments, each respective object is represented as a vector of terms. In VSM, the importance of a term is determined by its frequency of occurrence in an object. The similarity between any two vectors can be determined by calculating the dot product between the vectors and determining the cosine of the angle between the vectors. Non-limiting examples of text mining methods suitable for use in the present disclosure are described, for instance, in Anaya, “Comparing Latent Dirichlet Allocation and Latent Semantic Analysis as Classifiers,” Dissertation, December 2011, UNT, available on the Internet at digital.library.unt.edu/ark:/67531/metadc103284/, which is hereby incorporated herein by reference in its entirety.

In some embodiments, the determining makes use of a generative model, such as a topic model. Generative models seek to model the joint probability distribution on an observable variable X and a target variable Y. In such instances, the observable variable X is a continuous variable including the abundances of analytes measured for each capture spot in the set of capture spots from the tissue sample, and the target variable Y is a discrete variable consisting of a finite set of labels, or topics, such as for the classification of cell types. Non-limiting examples of topic models include Latent Semantic Analysis (LSA), probabilistic Latent Semantic Analysis (pLSA), Latent Dirichlet Allocation (LDA), and/or the correlated topics model (CTM). Advantageously, such methods can be used to resolve classification tasks involving synonymy (e.g., terms having similar or identical meaning) and/or polysemy (e.g., terms having membership in multiple class labels, such as where a particular gene is expressed in multiple cell types). More specifically, topic models are mixed-membership models, where objects are not assumed to belong to single topics (e.g., each capture spot corresponds to a single cell type) but can belong to several topics (e.g., each capture spot corresponds to multiple cells of multiple cell types). An example capture spot that encompasses cells of different cell types is illustrated in FIG. 3. Moreover, mixed-membership models consider instances in which the distributions or proportions of topics vary across objects (e.g., different capture spots have different cell type proportions) and each respective topic has a corresponding term distribution (e.g., each cell type has a particular gene expression profile).

Latent semantic analysis (LSA) utilizes singular value decomposition (SVD) to reduce a matrix d×t to a filtered matrix d′×t′. Because a first object represented as a first vector of terms can be compared to a second object represented as a second vector of terms using the dot product of vectors, the resulting reduced dimensional matrix allows for lower complexity and reduced computational resources in determining the distances and/or similarities between objects. In some instances, LSA is limited by the difficulty in determining an appropriate number of dimensions for use in dimension reduction. Moreover, the resulting vectors are generally orthogonal, such that LSA cannot be used effectively to resolve issues of polysemy.

Probabilistic latent semantic analysis (pLSA), or probabilistic latent sematic indexing (pLSI), utilizes a statistical model called an aspect model that assumes a “bag of words” context and generates the probability of each term occurring in an object p(w,d) independently. Interdependence between terms in an object is assumed to be explained by the latent topics to which each object belongs. In particular, the model assumes (i) a document d is generated with a probability p(d), (ii) a topic z is drawn with a probability p(z/d), and (iii) a word w is generated with a probability p(w/z). Then, the probability of selecting a term in an object is p(w,d)=Σ_{z} p(z) p(w/z) p(d/z). The objective of pLSA is to maximize a log likelihood function based on the probability p(w,d). Advantageously, pLSA allows for terms to appear in multiple topics, thus partially resolving polysemy.

Referring to block 212, in some embodiments, the determining makes use of a latent Dirichlet allocation model.

In natural language processing, Latent Dirichlet Allocation (LDA) is a generative statistical model that explains a set of observations (terms) through unobserved groups (topics), where each group explains similarities between portions of the data. Terms are collected into objects, and each term's presence is attributable to one of the object's topics. Each object will contain a small number of topics. More particularly, LDA is a Bayesian mixture model for discrete data where topics are assumed to be uncorrelated. The correlated topics model (CTM) is an extension of the LDA model where correlations between topics are allowed. As with pLSA, LDA is based on an aspect model.

In some embodiments, the determining includes inputting, as input, a matrix of dimensions objects d×terms t, or capture spots x analytes as illustrated by the example matrix 402 in FIG. 4. The total number of unique molecules, or total analyte expression, in a given capture spot is M_{d}. As a generative probabilistic model, for each object in a set of objects, the LDA model performs a series of steps. A term distribution β is determined for each topic using a uniform Dirichlet distribution with scaling parameter δ. The proportions θ of the topic distribution for the object are determined using the uniform Dirichlet distribution with scaling parameter α. For each term wᵢ in the plurality of terms, (i) a topic is selected zᵢ˜Multinomial(θ), and (ii) a term is selected wᵢ from a multinomial probability distribution conditioned on the topic zᵢ: p(wᵢ|zᵢ, β), where β is the term distribution of topics and contains the probability of a term occurring in a given topic.

LDA seeks to identify the posterior distribution of the latent parameters, θ and β, given the input data (e.g., the observed gene expression in the dataset). For instance, referring to block 214, in some embodiments, the determining refines the latent Dirichlet allocation model using expectation maximization.

In some instances, the probabilities for expectation maximization cannot be tractably computed. Thus, in some embodiments, the determining refines the latent Dirichlet allocation model using variational expectation-maximization (VEM). Alternatively or additionally, in some embodiments, the determining approximates LDA using a Markov chain Monte Carlo (MCMC) procedure, such as Gibbs sampling. Such methods predict future states as a condition of a current state, according to a state transition distribution.

LDA methods suitable for use in the present disclosure are further described, for example, in Anaya, “Comparing Latent Dirichlet Allocation and Latent Semantic Analysis as Classifiers,” Dissertation, December 2011, UNT, available on the Internet at digital.library.unt.edu/ark:/67531/metadc103284/; Grün and Hornik (2011), “topicmodels: An R Package for Fitting Topic Models,” Journal of Statistical Software, 40(13), 1-30, doi: 10.18637/jss.v040.i13; and Miller et al., “Reference-free cell type deconvolution of multi-cellular pixel-resolution spatially resolved transcriptomics data,” Nat Commun. 2022; 13(1):2339, each of which is hereby incorporated herein by reference in its entirety.

As indicated above, in some embodiments, terms refer to analytes (e.g., genes), topics refer to cell types, and objects refer to capture spots. Thus, in some embodiments, given a count matrix of gene expression in multi-cellular capture spots, LDA is used to determine the transcriptional profile for each cell type as well as the proportional representation of cell type in each capture spot. Consider the first current iteration 136-1 determined by setting the maximum number of proposed cell types 132 (e.g., Q cell types), illustrated in FIG. 1C. The analyte abundances of measured for each capture spot in the set of capture spots are used as input to a text mining model, such as LDA. This generates an analyte profile for each cell type in the Q cell types, where the analyte profile includes, for each respective analyte in the plurality of analytes, an abundance value of the respective analyte in the respective cell type. Then, for a first cell type 138-1-1 of current iteration 1 136-1, the corresponding analyte profile includes abundance values 139-1-1-1, . . . 139-1-1-K (e.g., pseudo-counts, relative proportions, etc.) for each of K analytes measured in the tissue sample that contribute to the first cell type. This is repeated for each of the proposed cell types in the Q cell types evaluated in the first current iteration, where any one cell type in the plurality of Q cell types has a same or different analyte profile from any other cell type in the plurality of Q cell types.

The proportional representation of cell type in each capture spot is illustrated by the example schematic in FIG. 3. For instance, Cell type 1 in the example capture spot is represented at a smaller proportion in the cells encompassed by capture spot compared to Cell type 2. Similarly, Cell type 3 is represented at a smaller proportion compared to Cell type 4. In some embodiments, the proportional representation of cell type in a respective capture spot is provided as a corresponding vector that includes, for each respective cell type in the plurality of Q cell types, the corresponding proportion (e.g., a ratio, a fraction, a percentage, etc.) of the cell type in the capture spot.

Generally, LDA is performed for a predefined number of topics, or cell types, where the predefined number of topics is set, e.g., by a user. In some implementations, the number of proposed cell types is based on the number of clusters obtained from an initial clustering method, such as the clustering of cells and/or capture spots described above (see, for example, the section entitled “Obtaining information”). In some implementations, the number of proposed cell types is the number of clusters obtained from an initial clustering method. In some implementations, the number of proposed cell types is the number of clusters obtained from an initial clustering method plus n, where n is a positive integer from 1 to 10. In some implementations, the number of proposed cell types is the number of clusters obtained from an initial clustering method minus n, where n is a positive integer from 1 to 10.

In some embodiments, the determining comprises obtaining a matrix dimensioned by a plurality of objects and a plurality of terms, where each respective object in the plurality of objects represents a corresponding capture spot in the set of capture spots, each respective term in the plurality of terms represents a corresponding analyte in the plurality of analytes, and the obtaining utilizes natural language processing to populate, for each respective object in the plurality of objects and for each respective term in the plurality of terms, a corresponding abundance for the respective analyte in the tissue sample, or a representation thereof, measured at the respective capture spot. In some embodiments, the natural language processing comprises a generative statistical model (e.g., LDA) that is further refined by a variational expectation-maximization or Markov chain Monte Carlo procedure.

In some embodiments, the method further includes, prior to the determining, removing one or more analytes from the plurality of analytes. In some embodiments, the method further includes, prior to the determining, removing one or more capture spots from the set of capture spots.

Referring to block 216, in some embodiments, the method further includes, prior to the determining, removing from the plurality of analytes those analytes that are present in more than a first threshold percentage of the set of capture spots, and removing from the plurality of analytes those analytes that are present in less than a second threshold percentage of the set of capture spots.

Referring to block 218, in some embodiments, the first threshold percentage is 95% and the second threshold percentage is 5%.

In some embodiments, the first threshold percentage is at least 70%, at least 75%, at least 80%, at least 85%, at least 90%, at least 95%, at least 98%, or at least 99%. In some embodiments, the first threshold percentage is no more than 100%, no more than 99%, no more than 98%, no more than 95%, no more than 90%, or no more than 80%. In some embodiments, the first threshold percentage is from 70% to 90%, from 80% to 95%, from 90% to 99%, or from 95% to 100%. In some embodiments, the first threshold percentage falls within another range starting no lower than 70% and ending no higher than 100%.

In some embodiments, the method further includes, prior to the determining, removing from the plurality of analytes those analytes that are present in 100% of the set of capture spots.

In some embodiments, the second threshold percentage is at least 1%, at least 2%, at least 3%, at least 4%, at least 5%, at least 10%, or at least 20%. In some embodiments, the second threshold percentage is no more than 30%, no more than 20%, no more than 10%, no more than 5% or no more than 3%. In some embodiments, the second threshold percentage is from 1% to 5%, from 2% to 10%, from 5% to 20%, or from 15% to 30%. In some embodiments, the second threshold percentage falls within another range starting no lower than 1% and ending no higher than 30%.

In some embodiments, the determining that a respective analyte is present in a respective percentage of capture spots is determined by matching the respective analyte to one or more respective barcodes for a corresponding one or more respective capture spots. Non-limiting examples of barcodes suitable for use in the present disclosure are described, for example, in United States Patent Publication No. US-2021-0155982-A1, entitled “Pipeline for Spatial Analysis of Analytes,” published May 27, 2021; International Patent Publication No. WO2021/102039, entitled “Pipeline for Spatial Analysis of Analytes,” published May 27, 2021; and U.S. Pat. No. 11,514,575B2, entitled “Systems and methods for identifying morphological patterns in tissue samples,” published Nov. 29, 2022, each of which is hereby incorporated by reference.

In some embodiments, the method further includes, prior to the determining, removing from the plurality of analytes those analytes that have less than a first threshold number of molecules captured from the tissue sample.

In some embodiments, the first threshold number of molecules is at least 1, at least 2, at least 3, at least 4, at least 5, at least 6, at least 7, at least 8, at least 9, at least 10, at least 20, at least 30, at least 40, at least 50, or at least 100 molecules. In some embodiments, the first threshold number of molecules is no more than 500, no more than 100, no more than 50, no more than 20, no more than 10, or no more than 5 molecules. In some embodiments, the first threshold number of molecules is from 1 to 10, from 2 to 20, from 5 to 50, from 40 to 100, or from 100 to 500 molecules. In some embodiments, the first threshold number of molecules falls within another range starting no lower than 1 molecule and ending no higher than 500 molecules.

In some embodiments, the determining the number of molecules for a respective analyte in the tissue sample is determined by measuring the number of unique molecular identifiers (UMIs) that correspond to the respective analyte in the input information. Non-limiting examples of unique molecular identifiers (UMIs) suitable for use in the present disclosure are described, for example, in United States Patent Publication No. US-2021-0155982-A1, entitled “Pipeline for Spatial Analysis of Analytes,” published May 27, 2021; International Patent Publication No. WO2021/102039, entitled “Pipeline for Spatial Analysis of Analytes,” published May 27, 2021; and U.S. Pat. No. 11,514,575B2, entitled “Systems and methods for identifying morphological patterns in tissue samples,” published Nov. 29, 2022, each of which is hereby incorporated by reference.

In some embodiments, the method further includes, prior to the determining, removing from the set of capture spots those capture spots that have less than a second threshold number of analytes detected at the respective capture spot.

In some embodiments, the second threshold number of molecules is at least 10, at least 20, at least 30, at least 40, at least 50, at least 60, at least 70, at least 80, at least 90, at least 100, at least 200, at least 300, at least 400, at least 500, or at least 1000 molecules. In some embodiments, the second threshold number of molecules is no more than 5000, no more than 1000, no more than 500, no more than 200, no more than 100, or no more than 50 molecules. In some embodiments, the second threshold number of molecules is from 10 to 100, from 20 to 200, from 50 to 500, from 400 to 1000, or from 1000 to 5000 molecules. In some embodiments, the second threshold number of molecules falls within another range starting no lower than 10 molecule and ending no higher than 5000 molecules.

In some embodiments, the removing is performed in a filtering step that generates a filtered matrix, such as matrix 402 illustrated in FIG. 4.

In some embodiments, as described above, the determining is performed for a first current iteration 136-1 of proposed cell types, where the first current iteration is set as the maximum number of proposed cell types. In some embodiments, the maximum number of proposed cell types is determined by a user. In some implementations, the maximum number of proposed cell types is determined based on the number of clusters obtained from an initial clustering method, such as the clustering of cells and/or capture spots described above (see, for example, the section entitled “Obtaining information”). In some implementations, the maximum number of proposed cell types is the number of clusters obtained from an initial clustering method. In some implementations, the maximum number of proposed cell types is the number of clusters obtained from an initial clustering method plus n, where n is a positive integer from 1 to 10. In some implementations, the maximum number of proposed cell types is the number of clusters obtained from an initial clustering method minus n, where n is a positive integer from 1 to 10.

Referring to block 220, in some embodiments, the maximum number of proposed cell types is between 8 and 40 proposed cell types.

In some embodiments, the maximum number of proposed cell types is between 5 and 100 proposed cell types.

In some embodiments, the maximum number of proposed cell types is at least 3, at least 4, at least 5, at least 6, at least 7, at least 8, at least 9, at least 10, at least 20, at least 30, at least 40, at least 50, at least 100, or at least 200 cell types. In some embodiments, the maximum number of proposed cell types is no more than 500, no more than 200, no more than 100, no more than 50, no more than 30, no more than 20, no more than 10, or no more than 5 cell types. In some embodiments, the maximum number of proposed cell types is from 1 to 10, from 1 to 5, from 2 to 4, from 1 to 30, from 3 to 20, from 10 to 50, from 40 to 200, or from 100 to 500 cell types. In some embodiments, the maximum number of proposed cell types falls within another range of cells starting no lower than 1 cell type and ending no higher than 500 cell types.

Referring to block 222, in some embodiments, each capture spot in at least 10 percent of the set of capture spots includes analyte data from between 1 and 20 proposed cell types in the current iteration of the plurality of proposed cell types.

In some embodiments, each capture spot in at least 10 percent of the set of capture spots includes analyte data from between 2 and 10 proposed cell types in the current iteration of the plurality of proposed cell types.

In some embodiments, each capture spot in at least 1, at least 5, at least 10, at least 20, at least 30, at least 40, at least 50, or at least 80 percent of the set of capture spots includes analyte data from at least 1, at least 5, at least 10, at least 20, or at least 50 proposed cell types in the current iteration of the plurality of proposed cell types. In some embodiments, each capture spot in at least 1, at least 5, at least 10, at least 20, at least 30, at least 40, at least 50, or at least 80 percent of the set of capture spots includes analyte data from no more than 100, no more than 50, no more than 20, no more than 10, or no more than 5 proposed cell types in the current iteration of the plurality of proposed cell types. In some embodiments, each capture spot in at least 1, at least 5, at least 10, at least 20, at least 30, at least 40, at least 50, or at least 80 percent of the set of capture spots includes analyte data from 1 to 10, from 2 to 30, from 5 to 40, or from 20 to 100 proposed cell types in the current iteration of the plurality of proposed cell types. In some embodiments, each capture spot in at least 1, at least 5, at least 10, at least 20, at least 30, at least 40, at least 50, or at least 80 percent of the set of capture spots includes analyte data from another range of proposed cell types starting no lower than 1 proposed cell type and ending no higher than 100 proposed cell types.

In some embodiments, each capture spot in no more than 100, no more than 60, no more than 50, no more than 30, no more than 20, no more than 10, or no more than 5 percent of the set of capture spots includes analyte data from at least 1, at least 5, at least 10, at least 20, or at least 50 proposed cell types in the current iteration of the plurality of proposed cell types. In some embodiments, each capture spot in no more than 100, no more than 60, no more than 50, no more than 30, no more than 20, no more than 10, or no more than 5 percent of the set of capture spots includes analyte data from no more than 100, no more than 50, no more than 20, no more than 10, or no more than 5 proposed cell types in the current iteration of the plurality of proposed cell types. In some embodiments, each capture spot in no more than 100, no more than 60, no more than 50, no more than 30, no more than 20, no more than 10, or no more than 5 percent of the set of capture spots includes analyte data from 1 to 10, from 2 to 30, from 5 to 40, or from 20 to 100 proposed cell types in the current iteration of the plurality of proposed cell types. In some embodiments, each capture spot in no more than 100, no more than 60, no more than 50, no more than 30, no more than 20, no more than 10, or no more than 5 percent of the set of capture spots includes analyte data from another range of proposed cell types starting no lower than 1 proposed cell type and ending no higher than 100 proposed cell types.

In some embodiments, each capture spot in from 1 to 10, from 5 to 30, from 10 to 40, from 20 to 50, or from 50 to 100 percent of the set of capture spots includes analyte data from at least 1, at least 5, at least 10, at least 20, or at least 50 proposed cell types in the current iteration of the plurality of proposed cell types. In some embodiments, each capture spot in from 1 to 10, from 5 to 30, from 10 to 40, from 20 to 50, or from 50 to 100 percent of the set of capture spots includes analyte data from no more than 100, no more than 50, no more than 20, no more than 10, or no more than 5 proposed cell types in the current iteration of the plurality of proposed cell types. In some embodiments, each capture spot in from 1 to 10, from 5 to 30, from 10 to 40, from 20 to 50, or from 50 to 100 percent of the set of capture spots includes analyte data from 1 to 10, from 2 to 30, from 5 to 40, or from 20 to 100 proposed cell types in the current iteration of the plurality of proposed cell types. In some embodiments, each capture spot in from 1 to 10, from 5 to 30, from 10 to 40, from 20 to 50, or from 50 to 100 percent of the set of capture spots includes analyte data from another range of proposed cell types starting no lower than 1 proposed cell type and ending no higher than 100 proposed cell types.

Additional methods for the determining the current iteration of a plurality of proposed cell types are further disclosed below. It will be understood that any of the embodiments for determining the current iteration of the plurality of proposed cell types disclosed elsewhere herein for any other method is similarly contemplated for use in the following approaches, or any substitutions, modifications, additions, deletions, and/or combinations thereof, as will be apparent to one skilled in the art.

In some embodiments, the determining makes use of a Naïve Bayes model. Naïve Bayes models suitable for use are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

In some embodiments, the determining makes use of a mixture model, such as a Gaussian mixture model. Mixture models are probabilistic models for representing the presence of subpopulations within an overall population. Mixture models are described, for instance, in McLachlan et al., Bioinformatics 18(3):413-422, 2002.

In some embodiments, the determining makes use of a hidden Markov model. Generally, hidden Markov models (HMMs) refer to models that describes a probability distribution over a plurality of sequences, such as a sequence of terms in an object. HMMs are composed of a number of “states” that correspond to positions, e.g., in a sequence of terms. Each state “emits” observable term identities according to term emission probabilities, and the states are connected by state transition probabilities. From an initial state, a sequence of states can be generated according to the state transition probabilities, generating an observable sequence of terms according to the term emission probability distribution of the respective state. The sequence of states represents a Markov chain, for which an underlying hidden sequence can be inferred from an alignment of the HMM to the observed sequence. Hidden Markov models are described, for instance, in Schliep et al., 2003, Bioinformatics 19(1):1255-i263.

In some embodiments, the determining makes use of linear discriminant analysis. Linear discriminant analysis, normal discriminant analysis (NDA), or discriminant function analysis can be a generalization of Fisher's linear discriminant, a method used in statistics, pattern recognition, and machine learning to find a linear combination of features that characterizes or separates two or more classes of objects or events.

Additional non-limiting examples of generative models for use in the determining include probabilistic context-free grammars, Bayesian networks, averaged one-dependence estimators, Boltzmann machines, variational autoencoders, generative adversarial networks, flow-based generative models, energy-based models, and/or diffusion models.

### Blocks 224 and 225—Performing an Iteration Procedure.

Referring to block 224, the method further includes, when the current iteration 136 of the plurality of proposed cell types 138 exceeds a minimum number 134 of proposed cell types, performing a procedure. A respective distance metric is determined between each proposed cell type 138 in the current iteration 136 of the plurality of proposed cell types based on the abundance value 139 for each analyte in the plurality of analytes for each proposed cell type in the current iteration of the plurality of proposed cell types. The current iteration 136 of the plurality of proposed cell types 138 is reformed by merging a first proposed cell type and a second proposed cell type having a smallest distance metric among all unique pairs of proposed cell types in the current iteration of the plurality of proposed cell types.

In some embodiments, each current iteration of the plurality of proposed cell types corresponds to a respective value of k, where k indicates the number of proposed cell types to be determined in the tissue sample based on the analyte abundances measured in the tissue sample for the set of capture spots. Thus, k is initially set at the maximum number of proposed cell types, and each iteration of the (i) determining and (ii) reforming procedure results in a telescoping value for k (or until a minimum number is reached).

Consider, for instance, the example iteration module 130 depicted in FIG. 1C. For the first current iteration of the plurality of proposed cell types 136-1, the number of proposed cell types k is set to the maximum number of proposed cell types Q. Accordingly, the determining the current iteration of a plurality of proposed cell types generates, for each respective cell type 138-1-1, . . . 138-1-Q in the plurality of Q cell types, the abundance value 139 (e.g., 139-1-1-1, . . . 139-1-1-K) for each analyte in the plurality of K analytes corresponding to the respective cell type. Then consider a minimum number of proposed cell types R, where R<Q. At least a first iteration of the procedure is performed, including the (i) determining a respective distance metric between each proposed cell type in the current iteration of the plurality of Q proposed cell types based on the abundance value for each analyte in the plurality of analytes for each proposed cell type in the current iteration of the plurality of proposed cell types, and (ii) reforming the current iteration of the plurality of Q proposed cell types by merging a first proposed cell type and a second proposed cell type having a smallest distance metric among all unique pairs of proposed cell types in the current iteration of the plurality of proposed cell types. The reforming (e.g., by merging two cell types in the plurality of Q cell types) results in one fewer proposed cell types in the plurality of cell types in the next current iteration of the plurality of proposed cell types, or Q−1. Then, the second current iteration of the plurality of proposed cell types 136-2, is set to k=Q−1, including, for each respective cell type 138-2-1, . . . 138-2-(Q−1) in the plurality of Q−1 cell types, the abundance value 139 (e.g., 139-2-1-1, . . . 139-2-1-K) for each analyte in the plurality of K analytes corresponding to the respective cell type.

In some embodiments, the procedure includes a clustering algorithm. In other words, after determining the first current iteration of the plurality of proposed cell types (e.g., obtaining the cell types using a text mining approach), an iterative clustering is performed on the cell types to obtain clusters of proposed cell types induced at various levels of granularity. In some embodiments, the clustering algorithm includes hierarchical clustering using average linkage and total variation distance between each of the proposed cell types in the current iteration of the plurality of proposed cell types.

Any one of a number of clustering techniques can be used, examples of which include, but are not limited to, hierarchical clustering, k-means clustering, and density-based clustering. In an embodiment, a hierarchical density-based clustering algorithm is used (referred to as HDBSCAN, see, e.g., Campello et al., 2015, “Hierarchical density estimates for data clustering, visualization, and outlier detection,” ACM Trans Knowl Disc Data, 10(1), 5). In another embodiment, a community detection-based cluster algorithm is used, such as Louvain clustering (see, e.g., Blondel et al., 2008, “Fast unfolding of communities in large networks,” J stat mech: theor exp, 2008(10), P10008). In yet another embodiment, Leiden clustering is used. See, e.g., Traag et al., (2019), “From Louvain to Leiden: guaranteeing well-connected communities,” Sci Rep 9:5233, doi: 10.1038/s41598-019-41695-z. In still another embodiment, a diffusion path algorithm is used.

Clustering algorithms suitable for use are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973.

Once a method for measuring “similarity” or “dissimilarity” between points in a dataset has been selected, clustering makes use of a criterion function that measures the clustering quality of any partition of the data. Partitions of the dataset that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques suitable for use as classifiers are disclosed in Kaufman and Roussccuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Exemplary clustering techniques that can be used as classifiers include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

In some embodiments, the clustering is supervised clustering. In some embodiments, the clustering comprises unsupervised clustering (e.g., with no preconceived number of clusters and/or no predetermination of cluster assignments).

In some embodiments, the clustering algorithm is a graph-based clustering algorithm. In some embodiments, the clustering algorithm is a Markov cluster algorithm. In some embodiments, the clustering algorithm is single-linkage clustering or k-means clustering. See, for example, Enright et al., 2002, “An efficient algorithm for large-scale detection of protein families” Nucleic Acids Research 30(7), pp. 1575-1584, which is hereby incorporated herein by reference in its entirety. In some embodiments, the clustering is a non-graph-based clustering.

In some embodiments, the distance is an average linkage, where the distance between two cell types is the average of all distances between the members (e.g., analytes in a corresponding plurality of analytes for a respective cell type) of the two cell types. Alternatively or additionally, in some embodiments, the distance is a total variation distance, where the distance is determined between two probability distributions (e.g., for each respective cell type in a pair of cell types, a respective probability distribution of analytes in the corresponding plurality of analytes for the respective cell type). In some embodiments, the distance is a Euclidean distance. In other embodiments, other distance metrics are used (e.g., Chebyshev distance, Mahalanobis distance, Manhattan distance, etc.).

Clustering methods are known in the art, including such non-limiting examples as described in United States Patent Publication No. US-2021-0155982-A1, entitled “Pipeline for Spatial Analysis of Analytes,” published May 27, 2021; and International Patent Publication No. WO2021/102039, entitled “Pipeline for Spatial Analysis of Analytes,” published May 27, 2021, each of which is hereby incorporated herein by reference in its entirety. In some embodiments, the clustering includes any of the clustering methods disclosed herein (see, for example, the section entitled “Obtaining information,” above).

In some embodiments, the minimum number of proposed cell types is between 2 and 10 proposed cell types.

In some embodiments, the minimum number of proposed cell types is at least 1, at least 2, at least 3, at least 4, at least 5, at least 6, at least 7, at least 8, at least 9, at least 10, at least 20, at least 30, at least 40, at least 50, or at least 100 cell types. In some embodiments, the minimum number of proposed cell types is no more than 200, no more than 100, no more than 50, no more than 30, no more than 20, no more than 10, no more than 5, or no more than 3 cell types. In some embodiments, the minimum number of proposed cell types is from 1 to 10, from 1 to 5, from 2 to 4, from 1 to 30, from 3 to 20, from 10 to 50, or from 40 to 200 cell types. In some embodiments, the minimum number of proposed cell types falls within another range of cells starting no lower than 1 cell type and ending no higher than 200 cell types.

In some embodiments, the minimum number of proposed cell types is less than the maximum number of proposed cell types.

Referring to block 225, the procedure is repeated until the current iteration of the plurality of proposed cell types matches the minimum number 134 of proposed cell types.

Referring again to FIG. 1C, the procedure is repeated for each current iteration of the plurality of proposed cell types while the current iteration k exceeds the minimum number of proposed cell types, as described above, and a final iteration of the procedure is performed when the current iteration of the number of proposed cell types k is R+1. Thus, the reforming (e.g., by merging two cell types in the plurality of Q cell types) results in one fewer proposed cell types in the plurality of cell types, or R.

Consider that the preceding (penultimate) current iteration to the final current iteration L has a value of k=R+1. The final iteration of the procedure is performed, including the (i) determining a respective distance metric between each proposed cell type in the current iteration of the plurality of R+1 proposed cell types based on the abundance value for each analyte in the plurality of analytes for each proposed cell type in the current iteration of the plurality of proposed cell types, and (ii) reforming the current iteration of the plurality of R+1 proposed cell types by merging a first proposed cell type and a second proposed cell type having a smallest distance metric among all unique pairs of proposed cell types in the current iteration of the plurality of proposed cell types. The reforming (e.g., by merging two cell types in the plurality of R+1 cell types) results in one fewer proposed cell types in the plurality of cell types in the next current iteration of the plurality of proposed cell types, or R. Then, the Lth current iteration of the plurality of proposed cell types 136-L, is set to k=R, including, for each respective cell type 138-L-1, . . . 138-L-R in the plurality of R cell types, the abundance value 139 (e.g., 139-L-1-1, . . . 139-L-1-K) for each analyte in the plurality of K analytes corresponding to the respective cell type. Because the current iteration k matches the minimum number of proposed cell types R after the final iteration, the procedure is stopped.

Here, the procedure is repeated Q-R times, and the total number of current iterations of the plurality of proposed cell types is L=Q−R+1. In other words, the number of values of k to be evaluated after the telescoping will be L=Q−R+1. Consider an example case where the maximum number of proposed cell types is Q=10 and the minimum number of proposed cell types is R=8. Then, the first current iteration 136-1 of the plurality of proposed cell types will be set to k=10 cell types. After a first iteration of the (i) determining and (ii) reforming procedure, the second current iteration 136-2 of the plurality of proposed cell types will be k=9 cell types. After a second iteration of the (i) determining and (ii) reforming procedure, the third current iteration 136-3 of the plurality of proposed cell types will be k=8 cell types. The procedure is repeated Q−R=10−8=2 times, and the number of current iterations is Q−R+1=10−8+1=3. Thus, output information will be generated for each of 3 current iterations and their corresponding values of k. After the second iteration of the procedure, the third current iteration of the plurality of proposed cell types is k=8 cell types, which matches the minimum number of proposed cell types R=8, and the procedure stops.

In some embodiments, the number of repeats is equal to the number of decreasing values of k between the maximum number of proposed cell types (e.g., Q), as disclosed above, and the minimum number of proposed cell types (e.g., R), as disclosed above. In some embodiments, the number of repeats is at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 50, at least 100, or at least 500. In some embodiments, the number of repeats is no more than 1000, no more than 500, no more than 100, no more than 50, no more than 30, no more than 25, no more than 20, no more than 15, no more than 10, no more than 9, no more than 8, no more than 7, no more than 6, no more than 5, or no more than 4. In some embodiments, the number of repeats is between 1 and 5, between 1 and 10, between 3 and 15, between 8 and 20, between 15 and 50, between 40 and 100, between 80 and 500, or between 400 and 1000. In some embodiments, the number of repeats falls within another range starting no lower than 1 and ending no higher than 1000.

Block 226—Determining output information.

Referring to block 226, the method further includes determining output information, for each respective current iteration 136 of the plurality of proposed cell types, for each respective proposed cell type 138 in the respective current iteration of the plurality of proposed cell types, for each respective capture spot 122 in the set of capture spots, providing a respective proportion of cells 152 in the respective capture spot having the respective proposed cell type.

As described above, in some embodiments, a respective current iteration of a plurality of proposed cell types (e.g., obtained using a text mining method, such as latent Dirichlet allocation and/or a hierarchical clustering approach) includes, for each respective capture spot in the set of capture spots, a proportional representation of cell types in the respective capture spot.

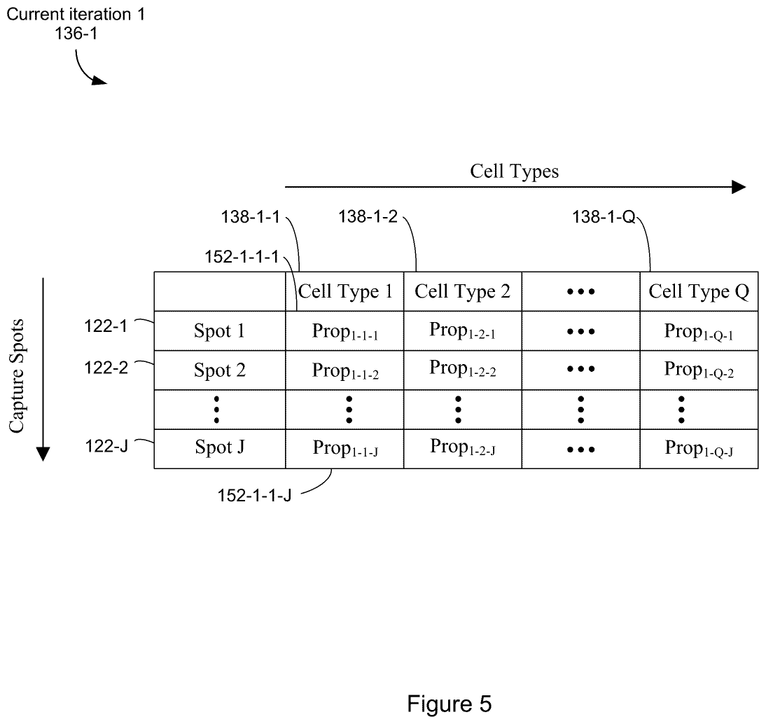

In some embodiments, for a given current iteration, the proportional representation of cell type in each capture spot, such as that illustrated by the example schematic in FIG. 3, is provided as a corresponding vector that includes, for each respective proposed cell type in the plurality of proposed cell types, the corresponding proportion (e.g., a ratio, a fraction, a percentage, etc.) of the cell type in the capture spot. FIGS. 1D and 5 illustrate. A first current iteration 136-1 in a plurality of current iterations (e.g., 136-1, 136-2, . . . 136-L) corresponds to a plurality of Q proposed cell types 138 (e.g., 138-1-1, 138-1-2, . . . 138-1-Q). The output information includes, for each capture spot in the set of capture spots 122 (e.g., 122-1, 122-2, . . . 122-J), a respective proportion of cells (“Prop”) 152 in the respective capture spot having the respective proposed cell type.

In some embodiments, for each respective capture spot in the set of capture spots, each respective proportion of cells is a ratio, a percentage, or a fraction. For instance, in some embodiments, for a respective capture spot in the set of capture spots, the proportions of cells over the plurality of proposed cell types are fraction values summing to 1.

In some embodiments, the proportional representation of cell type in a first capture spot (e.g., 122-1) is the same or different from the proportional representation of cell type in a second capture spot (e.g., 122-2). In some implementations, this is due to the capture of analytes at different spatial locations in the tissue sample having different mixtures of cells.

Alternatively or additionally, in some embodiments, for a respective capture spot, the proportional representation of cell type outputted in a first current iteration (e.g., 136-1) is the same or different from the proportional representation of cell type outputted for the same respective capture spot in a second current iteration (e.g., 136-2). In other words, in some implementations, the use of collapsing numbers of cell types by the iterative (i) determining and (ii) reforming procedure refines and/or optimizes the assignation of cell types to the analytes captured by a given capture spot from a portion of the tissue sample.

Referring to block 228, in some embodiments, the method further includes, for each respective current iteration of the plurality of proposed cell types, for each proposed cell type in the respective current iteration of the plurality of proposed cell types, providing a corresponding plurality of analytes in the proposed cell type and, for each analyte in the corresponding plurality of analytes, an abundance of the analyte.

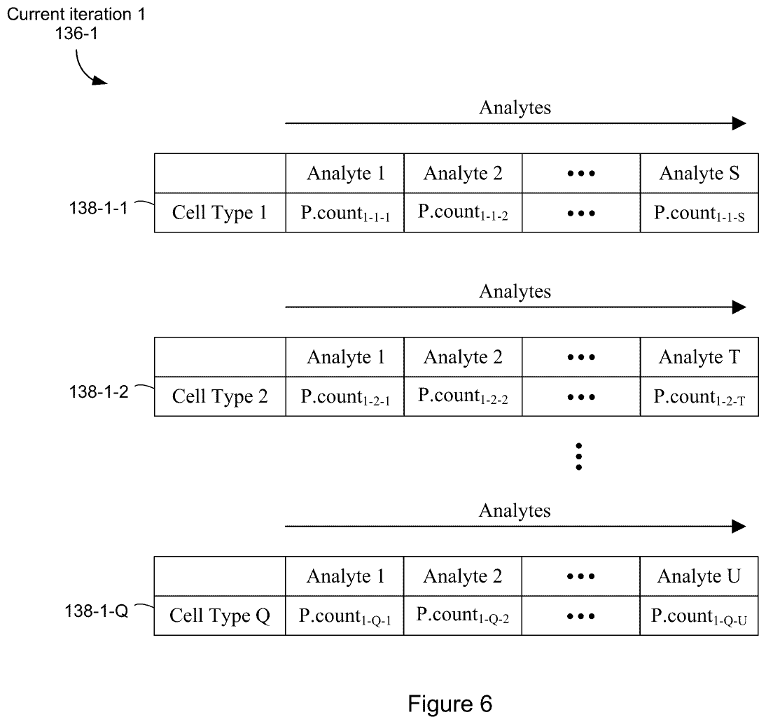

As described above, in some embodiments, each respective current iteration of the plurality of proposed cell types (e.g., obtained using a text mining method, such as latent Dirichlet allocation, and/or a hierarchical clustering approach) includes, for each proposed cell type in the plurality of proposed cell types, a corresponding analyte profile including an abundance, for the respective proposed cell type, of each respective analyte in the plurality of analytes in the respective cell type. As an example, in some embodiments, the analyte profile is a transcriptional profile for the respective cell type (e.g., a list of genes that characterizes the respective cell type, and a transcriptional abundance for each gene in the cell type). FIG. 6 illustrates.

Consider the first current iteration 136-1 determined by setting the maximum number of proposed cell types 132 (e.g., Q cell types), illustrated in FIG. 1C. Output from the determining step (e.g., using a text mining approach, such as LDA) generates an analyte profile for each cell type in the Q cell types, where the analyte profile includes a corresponding plurality of analytes in the proposed cell type and, for each respective analyte in the corresponding plurality of analytes, an abundance of the respective analyte in the respective cell type. Then, for a first cell type 138-1-1 of current iteration 1 136-1, the corresponding analyte profile includes a corresponding plurality of S analytes, each with a corresponding abundance (e.g., pseudo-counts, relative proportions, etc.) in a plurality of S abundances measured in the tissue sample that contribute to the first cell type. This is repeated for each of the proposed cell types in the Q cell types (e.g., 138-1-2, . . . 138-1-Q) evaluated in the first current iteration, where any one cell type in the plurality of Q cell types has a same or different analyte profile from any other cell type in the plurality of Q cell types.

In particular, in some embodiments, a first cell type (e.g., 138-1-1) has a same or different number of analytes in the corresponding plurality of analytes as a second cell type (e.g., 138-1-2). For instance, as illustrated in FIG. 6, a first cell type 138-1-1 having a corresponding plurality of S analytes, a second cell type 138-1-2 having a corresponding plurality of T analytes, and a Q^{th} cell type having a corresponding plurality of U analytes are similarly contemplated.

Alternatively or additionally, in some embodiments, for a respective cell type, the corresponding plurality of analytes, and/or their abundances thereof, outputted in a first current iteration (e.g., 136-1) is the same or different from the corresponding plurality of analytes and/or abundances outputted for the same respective cell type in a second current iteration (e.g., 136-2). In other words, in some implementations, the approach of collapsing the numbers of cell types by the iterative (i) determining and (ii) reforming procedure refines and/or optimizes the determination and characterization of cell types based on analytes determined to be associated with the cell types and their corresponding analyte abundances captured from the tissue sample.

In some embodiments, each respective abundance is a pseudo-count of the respective analyte in the proposed cell type. In some embodiments, pseudo-counts refer to counts of the analytes proportional to their correspondence to the proposed cell type, rather than total counts within the tissue sample or within each capture spot. Alternatively or additionally, in some such embodiments, pseudo-counts reflect the generated probability of the analytes in the corresponding plurality of analytes, according to the output of the cell type determination process (e.g., text mining and/or hierarchical clustering).

In some embodiments, each respective abundance is a relative proportion of the respective analyte relative to all other analytes in the corresponding plurality of analytes for the proposed cell type. For instance, in some embodiments, the relative proportion is a ratio, a percentage, or a fraction. For instance, in some embodiments, for a respective cell type in the plurality of proposed cell types, the relative proportions of analytes over the corresponding plurality of analytes for the respective cell type are fraction values summing to 1.

In some embodiments, the method further includes, for each respective current iteration of the plurality of proposed cell types, for each respective capture spot in the set of capture spots, ranking the plurality of proposed cell types by the corresponding proportion of cells in the respective capture spot. In some embodiments, the method further includes, for each respective current iteration of the plurality of proposed cell types, for each respective capture spot in the set of capture spots, assigning, to the respective capture spot, the respective cell type in the plurality of proposed cell types having the highest proportion of cells.

In some embodiments, the method further includes, for a respective current iteration of the plurality of proposed cell types, for each proposed cell type in the respective current iteration of the plurality of proposed cell types, ranking the corresponding plurality of analytes by their respective abundance. In some implementations, the method further includes for the respective current iteration, for each proposed cell type in the respective current iteration of the plurality of proposed cell types, selecting a subset of top N analytes having the top N highest abundances. In some embodiments, N is at least 1, at least 5, at least 10, at least 20, at least 30, at least 50, or at least 80. In some embodiments, N is no more than 100, no more than 50, no more than 30, no more than 20, or no more than 10. In some embodiments, N is from 1 to 10, from 1 to 20, from 10 to 50, from 30 to 80, or from 50 to 100. In some embodiments, N falls within another range starting no lower than 1 and ending no higher than 100.

Referring to block 230, in some embodiments, the method further includes overlaying, for each capture spot in the set of capture spots, an indication of a respective proportion of cells in the respective capture spot having a proposed cell type in the current iteration of the plurality of proposed cell types.

In particular, in some such embodiments, the method further includes, for each respective current iteration of the plurality of proposed cell types, for each respective cell type in the plurality of proposed cell types, overlaying, for each capture spot in the set of capture spots, an indication of the respective proportion of cells in the respective capture spot having the respective proposed cell type.

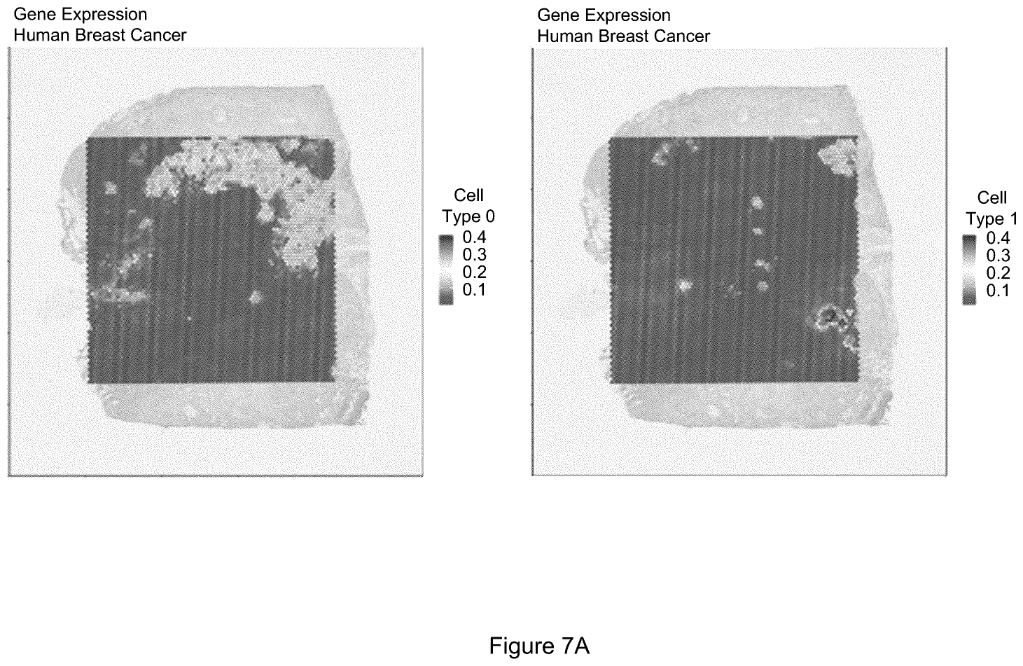

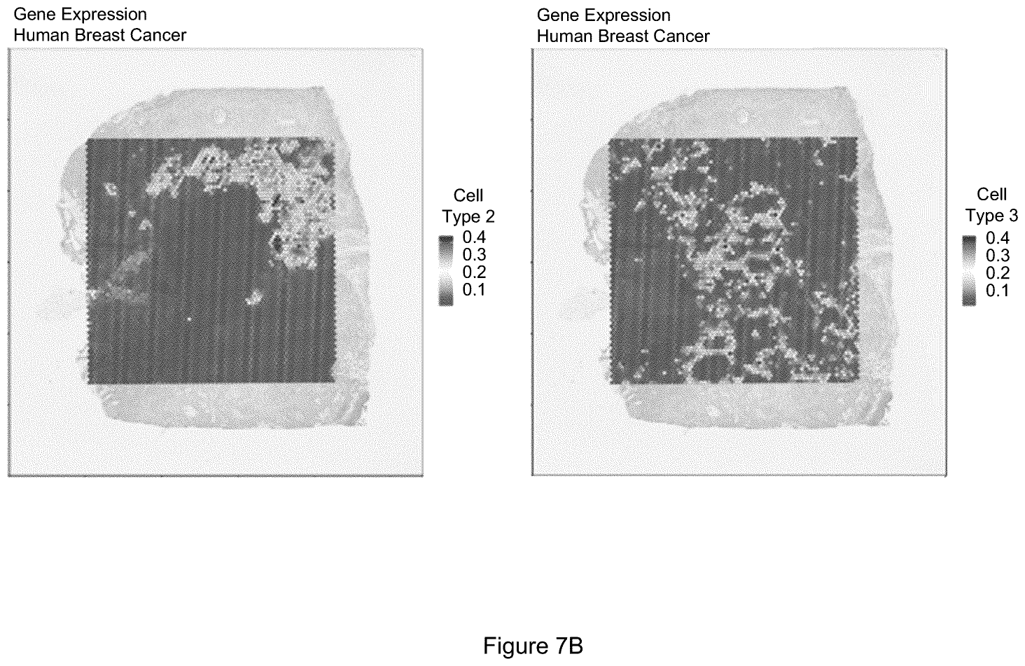

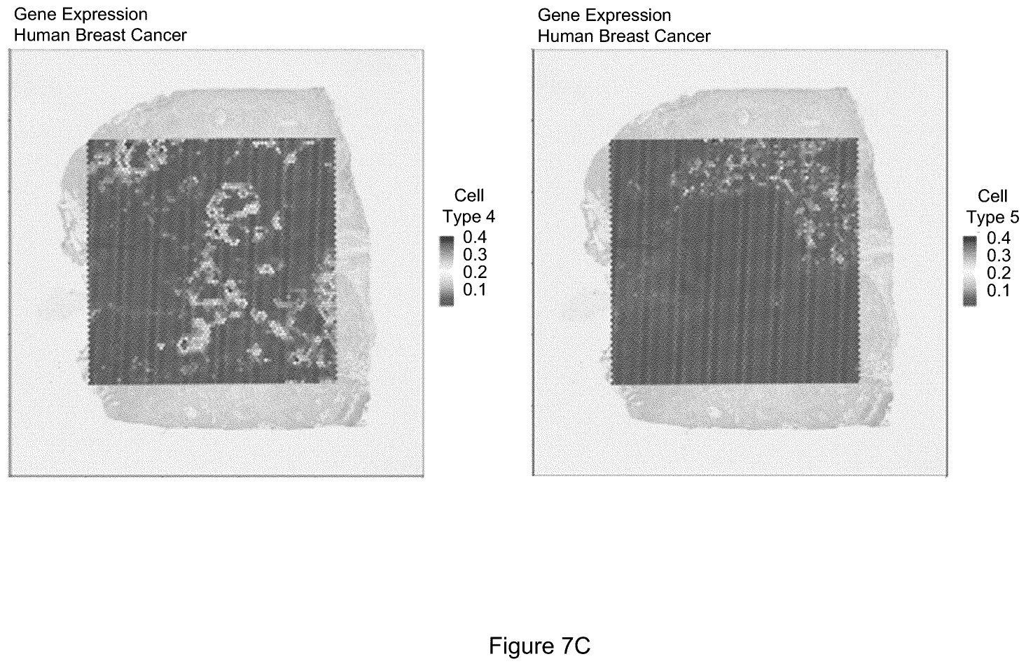

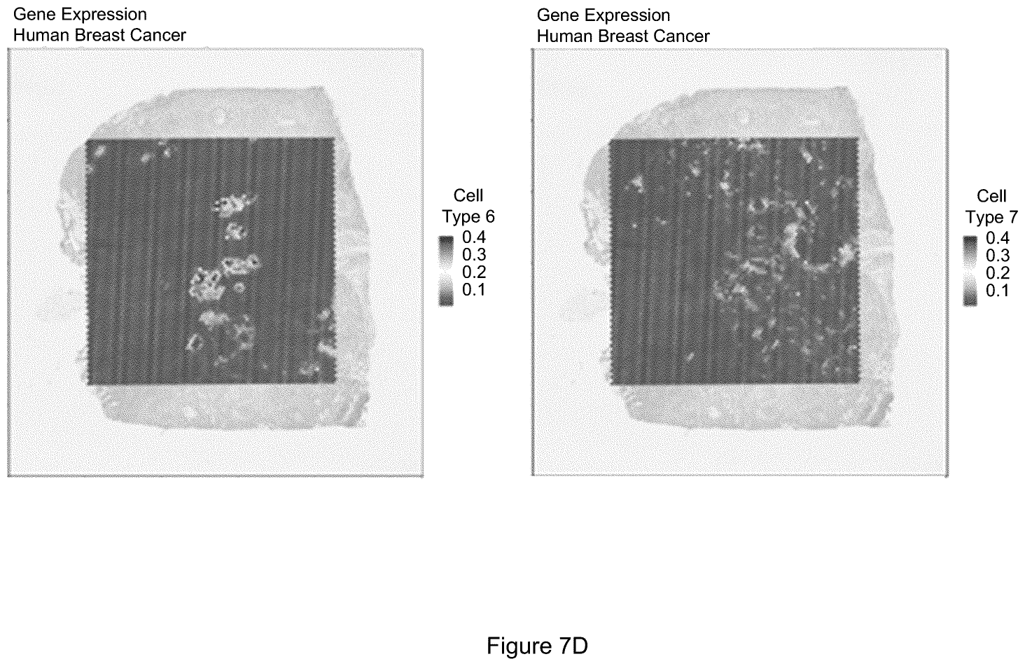

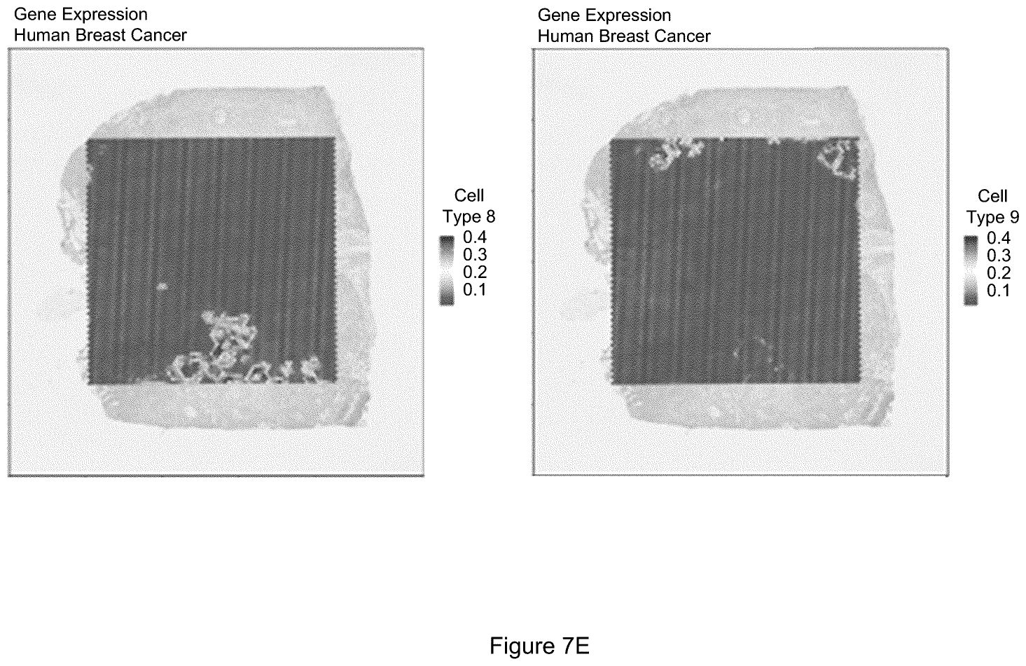

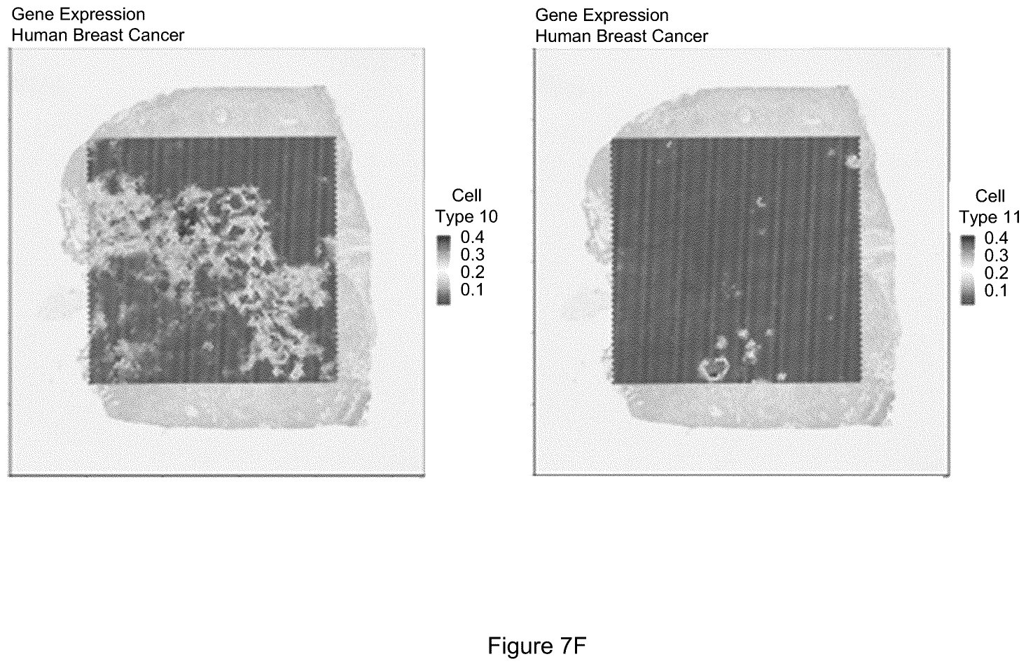

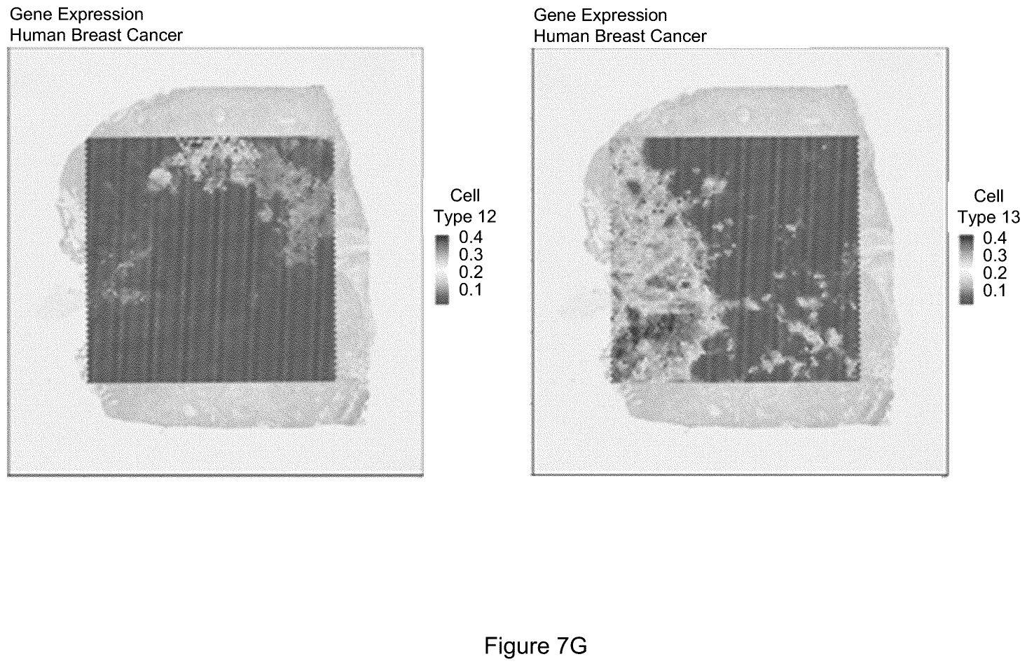

FIGS. 7A-G illustrate example spatial heat maps in which each capture spot in the set of capture spots is overlaid with an indication of the respective proportion of cells in the respective capture spot, where each respective heat map corresponds to a different proposed cell type in the plurality of proposed cell types, for a given current iteration (e.g., 14 proposed cell types). The relative proportions of cells having the respective cell type are indicated by the legends (e.g., 0.1, 0.2, 0.3, 0.4) and are visualized as intensity of color.

In some embodiments, the overlay is displayed on a display, such as a display in visualization system 100.

In some embodiments, the capture spots and/or the overlay are further overlaid on the image of the tissue sample.

In some embodiments, the indication of the respective proportion of cells in the respective capture spot having the respective proposed cell type is a differentiation of color, shade, or pixel intensity (e.g., the greater the proportion of cells in the capture spot, the more intensely the capture spot will be colored in the heatmap).

In some embodiments, the method further includes, for each respective current iteration of the plurality of proposed cell types, for each respective capture spot in the set of capture spots, assigning, to the respective capture spot, the respective cell type in the plurality of proposed cell types having the highest proportion of cells; and overlaying, for each respective capture spot in the set of capture spots, an indication of the cell type assigned to the respective capture spot based on the assigning.

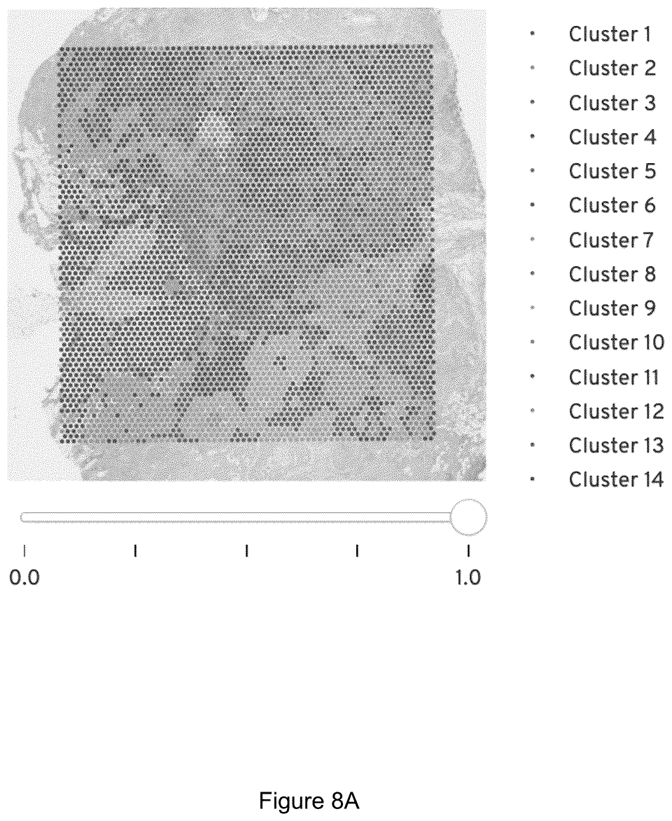

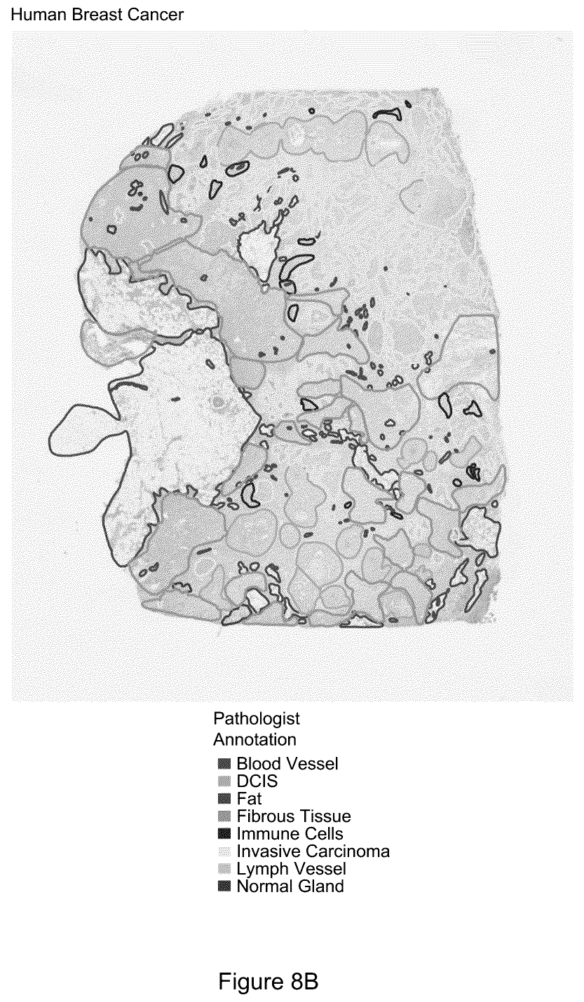

FIG. 8A illustrates an example spatial visualization in which each capture spot in the set of capture spots is overlaid with an indication of a cell type that is assigned to the respective capture spot, for a given current iteration (e.g., 14 proposed cell types; “Cluster”).

In some embodiments, the method further includes, for each respective current iteration of the plurality of proposed cell types, validating the overlay using pathologist annotations of the image of the tissue sample.

Various non-limiting methods for visualizing analyte data, including spatial analyte data, images of tissue samples, capture spots, clusters, and/or overlays, are further described in United States Patent Publication No. US-2021-0155982-A1, entitled “Pipeline for Spatial Analysis of Analytes,” published May 27, 2021; and International Patent Publication No. WO2021/102039, entitled “Pipeline for Spatial Analysis of Analytes,” published May 27, 2021, each of which is hereby incorporated herein by reference in its entirety.

In some embodiments, the method further includes using the output information to determine an identity of a respective cell type in the plurality of proposed cell types. For instance, in some embodiments, the method further includes, for a respective proposed cell type, using the corresponding plurality of analytes and their corresponding abundances in the proposed cell type (as shown in FIG. 6) to determine the identity of the cell type.

In some embodiments, the determining the identity is performed by comparing one or more analytes in the corresponding plurality of analytes and/or one or more abundances thereof to experimentally verified biological pathways and/or gene expression profiles for different cell types.

Referring to block 231, in some embodiments, the method further includes using the output information to determine whether or not the subject has a condition.

Referring to block 232, in some embodiments, the method further includes providing a treatment of the subject when it is determined that the subject has the condition.

Referring to block 234, in some embodiments, the treatment comprises a composition comprising a small molecule compound and one or more excipient and/or one or more pharmaceutically acceptable carrier and/or one or more diluent.

In some embodiments, the small molecule compound has a molecular weight of 2000 Daltons or less.

In some embodiments, the small molecule compound satisfies any two or more rules, any three or more rules, or all four rules of Lipinski's rule of Five: (i) not more than five hydrogen bond donors, (ii) not more than ten hydrogen bond acceptors, (iii) a molecular weight under 500 Daltons, and (iv) a Log P under 5.

Referring to block 236, in some embodiments, the condition is inflammation or pain.

Referring to block 238, in some embodiments, the condition is a disease.

In some embodiments, the condition is asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersinia pestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection.

In some embodiments, the condition is cancer.

In some implementations, the systems and methods disclosed herein reveal cellular context and interactions within a tissue sample that would not be otherwise discernable. This leads to the discovery of relationships between (A) aspects of the cellular phenotypes, such as genome (e.g., genomic rearrangements, structural variants, copy number variants, single nucleotide polymorphisms, loss of heterozygosity, rare variants), epigenome (e.g., DNA methylation, histone modification, chromatin assembly, protein binding), transcriptome (e.g., gene expression, alternative splicing, non-coding RNAs, small RNAs), proteome (e.g., protein abundance, protein-protein interactions, cytokine screening), metabolome (e.g., absence, presence, or amount of small molecules, drugs, metabolites, and lipids), and/or phenome (e.g., functional genomics, genetics screens, morphology), and/or (B) particular phenotypic states, such as absence or presence of a marker, participation in a biological pathway, disease state, and/or absence or presence of a disease state, to name a few non-limiting examples. The determination of cell types within the sample allows for taking an action with respect to the sample or with respect to a source of the sample. For example, depending on a distribution of cell types within a biological sample that is a tumor biopsy obtained from a subject, a specific treatment can be selected and administered to the subject.

### ADDITIONAL EMBODIMENTS

Another aspect of the present disclosure provides a computer system including one or more processors, memory, and one or more programs. The one or more programs are stored in the memory and are configured to be executed by the one or more processors, the one or more programs for determining cell type, the one or more programs including instructions. The one or more programs for determining cell type include obtaining, in electronic form, input information for a set of capture spots, the input information including, for each capture spot in the set of capture spots, a corresponding position in an image of a tissue sample from a subject, and a respective abundance of each analyte in a plurality of analytes measured for each capture spot in the set of capture spots from the tissue sample. A current iteration of a plurality of proposed cell types that is set to a maximum number of proposed cell types is determined using the respective abundance of each analyte in the plurality of analytes measured for each capture spot in the set of capture spots from the tissue sample, where each respective cell type in the current iteration of the plurality of proposed cell types has an abundance value for each analyte in the plurality of analytes. When the current iteration of the plurality of proposed cell types exceeds a minimum number of proposed cell types, a procedure is performed including determining a respective distance metric between each proposed cell type in the current iteration of the plurality of proposed cell types based on the abundance value for each analyte in the plurality of analytes for each proposed cell type in the current iteration of the plurality of proposed cell types, and reforming the current iteration of the plurality of proposed cell types by merging a first proposed cell type and a second proposed cell type having a smallest distance metric among all unique pairs of proposed cell types in the current iteration of the plurality of proposed cell types. The procedure is repeated until the current iteration of the plurality of proposed cell types matches the minimum number of proposed cell types. Output information is determined, for each respective current iteration of the plurality of proposed cell types, for each respective proposed cell type in the respective current iteration of the plurality of proposed cell types, for each respective capture spot in the set of capture spots, providing a respective proportion of cells in the respective capture spot having the respective proposed cell type.

Another aspect of the present disclosure provides a computer readable storage medium storing one or more programs, the one or more programs comprising instructions, which when executed by an electronic device with one or more processors and a memory cause the electronic device to determine cell type by a method. The method includes obtaining, in electronic form, input information for a set of capture spots, the input information including, for each capture spot in the set of capture spots, a corresponding position in an image of a tissue sample from a subject, and a respective abundance of each analyte in a plurality of analytes measured for each capture spot in the set of capture spots from the tissue sample. A current iteration of a plurality of proposed cell types that is set to a maximum number of proposed cell types is determined using the respective abundance of each analyte in the plurality of analytes measured for each capture spot in the set of capture spots from the tissue sample, where each respective cell type in the current iteration of the plurality of proposed cell types has an abundance value for each analyte in the plurality of analytes. When the current iteration of the plurality of proposed cell types exceeds a minimum number of proposed cell types, a procedure is performed including determining a respective distance metric between each proposed cell type in the current iteration of the plurality of proposed cell types based on the abundance value for each analyte in the plurality of analytes for each proposed cell type in the current iteration of the plurality of proposed cell types, and reforming the current iteration of the plurality of proposed cell types by merging a first proposed cell type and a second proposed cell type having a smallest distance metric among all unique pairs of proposed cell types in the current iteration of the plurality of proposed cell types. The procedure is repeated until the current iteration of the plurality of proposed cell types matches the minimum number of proposed cell types. Output information is determined, for each respective current iteration of the plurality of proposed cell types, for each respective proposed cell type in the respective current iteration of the plurality of proposed cell types, for each respective capture spot in the set of capture spots, providing a respective proportion of cells in the respective capture spot having the respective proposed cell type.

### EXAMPLES

### Example 1-Reference-Free Deconvolution Method Using a Natural Language Processing-Based Approach

A reference-free spatial deconvolution approach was performed to determine cell type in a tissue sample, in accordance with an embodiment of the present disclosure.

### Sample Preparation Methods

Briefly, from a single tumor biopsy, fresh frozen data was acquired using the 3′ and 5′ gene expression single-cell assays. A section of the same tumor block was fixed to assay both the single cells, using a Fixed RNA Profiling solution (FRP), and the spatial context of the transcriptome using a spatial transcriptomics analysis pipeline (Visium CytAssist) for formalin-fixed, paraffin-embedded (FFPE).

Samples and sample collection. A single FFPE breast cancer tissue block (TNM stage T2N1M0, ER+/HER2+/PR−) was collected. Corresponding dissociated tumor cells, fresh frozen in liquid nitrogen, were also sampled from the same biopsy (patient matched). 5 μm sections were taken from the FFPE tissue using a microtome (Thermo Scientific HM355S; MX35 blades). For the Fixed RNA Profiling (scFFPE-seq) workflow, 25 μm FFPE curls were collected into a tube prior to serial sectioning for the spatial transcriptomics analysis (two replicates of 5 μm sections), then an additional 25 μm FFPE curl was collected into the same tube reserved for scFFPE-seq. These pooled 25 μm curls (50 μm total) were treated as a single replicate.

Fixed RNA Profiling (scFFPE-seq). The scFFPE-seq data was produced in order to precisely define the cell types present in serial tissue sections. After dissociation, approximately 600,000 cells were washed, counted and resuspended. Sequencing libraries were generated, and libraries were sequenced on an Illumina NovaSeq with paired-end dual-indexing. Sequencing libraries were demultiplexed and sequencing files were processed (Cell Ranger v7.0.1; 10× Genomics) as described in Janesick et al., “High resolution mapping of the breast cancer tumor microenvironment using integrated single cell, spatial and in situ analysis of FFPE tissue,” bioRxiv. 2022, doi: 10.1101/2022.10.06.510405, which is hereby incorporated herein by reference in its entirety. 3′ and 5′ GEX data were further collected from dissociated tumor cells to benchmark performance against the scFFPE-seq data.

Whole transcriptome spatial data. The whole transcriptome spatial data was produced in order to obtain whole transcriptome, spatially barcoded sequence data for a tissue section, where spatial barcodes corresponded to capture spots in a set of capture spots. A tissue section was imaged, followed by hematoxylin de-staining and de-crosslinking. A substrate with the tissue section was processed to transfer analytes to a spatial gene expression slide with a 0.42 cm2 capture area (Visium CytAssist). The probe extension and library construction steps follow an FFPE workflow. Libraries were sequenced with paired-end dual-indexing and sequencing libraries were demultiplexed. A spatial analysis pipeline (Space Ranger pipeline v2022.0705.1; 10× Genomics) was performed, thus obtaining, for each spot in the set of capture spots, a position in the tissue image and corresponding analyte abundances measured from the tissue for the respective spot. Moreover, the spatial analysis pipeline was used to obtain initial clusters using the analyte abundances obtained from the sequencing.

Further details regarding sample preparation, processing, and sequencing are described in Janesick et al., which is hereby incorporated herein by reference in its entirety.

### Deconvolution Methods.

After processing, the single-cell data (3′ and 5′ GEX and scFFPE-seq) and spatial data (whole transcriptome spatial data) were integrated. First, a plurality of cell types was classified using abundance data obtained from the single-cell 3′ and 5′ GEX assays. The single-cell, cell-type profiles were used to perform reference-guided deconvolution of capture spots in the spatial data. Deconvolution profiles (e.g., cell type profiles) in spatial data were observed to be similar when using either 3′ or 5′ single-cell assays as references. This allowed accurate identification and differentiation of mixtures of invasive carcinoma, ductal carcinoma in situ, immune, and stromal compartments. Using the FRP results as the reference, a more refined deconvolution profile was generated which enabled additional classification of multiple different cellular subtypes including myoepithelial, macrophages, invasive tumor, and dendritic cells.

To generate deconvolution profiles for spatial transcriptomics results without a companion single-cell dataset, a reference-free deconvolution method was performed as described in the present disclosure. In particular, the method included a natural language processing-based approach coupled with building a tree of relationships between cell types via hierarchical clustering. More specifically, the initial clusters obtained using the spatial analysis pipeline were used to set the maximum number of proposed cell types for the first current iteration of cell types, where each cell type included an abundance value for each analyte. An iterative procedure was performed until the current iteration matched a minimum number of proposed cell types. The procedure included (i) determining a distance metric between each cell type in the current iteration based on the analyte abundance values for each cell type and (ii) reforming the current iteration by merging a first and second cell type having a smallest distance metric. For each current iteration, output was determined including, for each cell type, for each spot, a proportion of cells in the spot having the cell type.

### Results.

The reference-free deconvolution method achieved accuracy equivalent to the identification of cellular subclasses using the reference guided analyses.

Moreover, cell types determined for a tissue sample using the presently disclosed methods compared against pathologist annotations of the same tissue sample showed good concordance in identified cell types. For instance, FIGS. 7A-G show example images including, for a respective current iteration of 14 proposed cell types, for each respective cell type in the 14 proposed cell types, for each capture spot in a set of capture spots, an overlay indicating a respective proportion of cells in the respective capture spot having the proposed cell type. FIG. 8A shows an aggregate overlay of the tissue sample where each capture spot in the set of capture spots has an indication of a corresponding proposed cell type. A comparison of the overlays in FIGS. 7A-G and 8A to the pathologist annotations in FIG. 8B reveals similarities in the classification of various portions of the image, indicating that the reference-free deconvolution approach disclosed in the present disclosure is capable of accurately identifying cell types, and proportions thereof, with spatial context in tissue samples. Taken together, these results help gain new insights into tumor biology by extending spatial datasets towards single cell resolution.

### CONCLUSION

All references cited herein are incorporated herein by reference in their entirety and for all purposes to the same extent as if each individual publication or patent or patent application was specifically and individually indicated to be incorporated by reference in its entirety for all purposes.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the present disclosure. As used in the description of the present disclosure and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (“or” in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method for determining cell type, the method comprising:
at a computer system comprising one or more processing cores and a memory:
A) obtaining, in electronic form, input information for a set of capture spots, the input information comprising:
for each capture spot in the set of capture spots, a corresponding position in an image of a tissue sample, and
a respective abundance of each analyte in a plurality of analytes measured for each capture spot in the set of capture spots from the tissue sample;
B) determining a current iteration of a plurality of proposed cell types that is set to a maximum number of proposed cell types using the respective abundance of each analyte in the plurality of analytes measured for each capture spot in the set of capture spots from the tissue sample, wherein each respective cell type in the current iteration of the plurality of proposed cell types has an abundance value for each analyte in the plurality of analytes;
C) when the current iteration of the plurality of proposed cell types exceeds a minimum number of proposed cell types, performing a procedure comprising:
determining a respective distance metric between each proposed cell type in the current iteration of the plurality of proposed cell types based on the abundance value for each analyte in the plurality of analytes for each proposed cell type in the current iteration of the plurality of proposed cell types, and
reforming the current iteration of the plurality of proposed cell types by merging a first proposed cell type and a second proposed cell type having a smallest distance metric among all unique pairs of proposed cell types in the current iteration of the plurality of proposed cell types;
D) repeating the procedure C) until the current iteration of the plurality of proposed cell types matches the minimum number of proposed cell types; and
E) determining output information, for each respective current iteration of the plurality of proposed cell types,
for each respective proposed cell type in the respective current iteration of the plurality of proposed cell types,
for each respective capture spot in the set of capture spots, providing a respective proportion of cells in the respective capture spot having the respective proposed cell type.

2. The method of claim 1, the method further comprising:
F) for each respective current iteration of the plurality of proposed cell types,
for each proposed cell type in the respective current iteration of the plurality of proposed cell types,
providing a corresponding plurality of analytes in the proposed cell type and, for each analyte in the corresponding plurality of analytes, an abundance of the analyte.

3. The method of claim 1, the method further comprising overlaying, for each capture spot in the set of capture spots, an indication of a respective proportion of cells in the respective capture spot having a proposed cell type in the current iteration of the plurality of proposed cell types.

4. The method of claim 1, wherein a capture spot in the set of capture spots comprises a capture domain, and wherein each capture spot in the set of capture spots is attached directly or indirectly to a substrate.

5. The method of claim 4, wherein a capture spot in the set of capture spots comprises a cleavage domain.

6. (canceled)

7. The method of claim 1, wherein the plurality of analytes comprises five or more analytes, ten or more analytes, fifty or more analytes, one hundred or more analytes, five hundred or more analytes, 1000 or more analytes, 2000 or more analytes, or between 2000 and 100,000 analytes.

8. The method of claim 1, wherein each capture spot in the set of capture spots has a unique spatial barcode that encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10^{12}.}

9. The method of claim 8, wherein each respective capture spot in the set of capture spots includes 1000 or more capture probes, 2000 or more capture probes, 10,000 or more capture probes, 100,000 or more capture probes, 1×10⁶ or more capture probes, 2×10⁶ or more capture probes, or 5×10⁶ or more capture probes.

10. The method of claim 9, wherein each capture probe in the respective capture spot includes a poly-A sequence or a poly-T sequence and the unique spatial barcode that characterizes the respective capture spot.

11.-13. (canceled)

14. The method of claim 1, wherein
a respective capture spot in the set of capture spots includes a respective plurality of capture probes, wherein:
each capture probe in the plurality of capture probes includes a capture domain that is characterized by a capture domain type in a plurality of capture domain types,
the plurality of capture domain types comprises between 5 and 15,000 capture domain types, and
the respective plurality of capture probes includes at least 5, at least 10, at least 100, or at least 1000 capture probes for each capture domain type in the plurality of capture domain types, and
each respective capture domain type in the plurality of capture domain types is configured to bind to a different analyte in the plurality of analytes.

15. (canceled)

16. The method of claim 1, wherein a respective capture spot in the set of capture spots includes a plurality of capture probes, wherein each capture probe in the plurality of capture probes includes a capture domain that is characterized by a single capture domain type configured to bind to each analyte in the plurality of analytes in an unbiased manner.

17. The method of claim 1, wherein each respective capture spot in the set of capture spots is contained within a 100 micron by 100 micron square on the substrate.

18. The method of claim 1, wherein a distance between a center of each respective capture spot to a neighboring capture spot in the set of capture spots on the substrate is between 10 microns and 100 microns.

19. The method of claim 1, wherein a shape of each capture spot in the set of capture spots on the substrate is a circular closed-form shape having a diameter of between 30 microns and 65 microns, and wherein a distance between a center of each respective capture spot to a neighboring capture spot in the set of capture spots on the substrate is between 50 microns and 80 microns.

20.-23. (canceled)

24. The method of claim 1, the method further comprising using the output information to determine whether or not the tissue sample was obtained from a subject having a condition, and providing a treatment of the subject when it is determined that the subject has the condition.

25. (canceled)

26. The method of claim 24, wherein the treatment comprises a composition comprising a small molecule compound and one or more excipient and/or one or more pharmaceutically acceptable carrier and/or one or more diluent, wherein the small molecule compound has a molecular weight of 2000 Daltons or less.

27.-28. (canceled)

29. The method of claim 24, wherein the condition is inflammation, pain, asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersinia pestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection.

30.-31. (canceled)

32. The method of claim 1, wherein the B) determining makes use of a latent Dirichlet allocation model and refines the latent Dirichlet allocation model using expectation maximization.

33. (canceled)

34. The method of claim 1, the method further comprising, prior to the determining,
removing from the plurality of analytes those analytes that are present in more than 95% of the set of capture spots, and
removing from the plurality of analytes those analytes that are present in less than 5% of the set of capture spots.

35. (canceled)

36. The method of claim 1, wherein each analyte in the plurality of analytes is a different gene, protein, mRNA, genomic DNA, intracellular protein, metabolite, or V(D)J sequence.

37. The method of claim 1, wherein the set of capture spots comprises 1000, 2000, 3000, 4000, 5000, 6000, 7000, 8000, 9000, or 10000 capture spots.

38. (canceled)

39. The method of claim 1, wherein the plurality of analytes comprises 100, 200, 300, 400, 500, or 1000 analytes.

40. The method of claim 1, wherein;
the maximum number of proposed cell types is between 8 and 40 proposed cell types,
the minimum number of proposed cell types is between 2 and 10 proposed cell types,
each capture spot in at least 10 percent of the set of capture spots includes analyte data from between 1 and 20 proposed cell types in the current iteration of the plurality of proposed cell types.

41.-45. (canceled)

46. A computer system comprising:
one or more processors;
memory; and
one or more programs, wherein the one or more programs are stored in the memory and are configured to be executed by the one or more processors, the one or more programs for determining cell type, the one or more programs including instructions for:
A) obtaining, in electronic form, input information for a set of capture spots, the input information comprising:
for each capture spot in the set of capture spots, a corresponding position in an image of a tissue sample, and
a respective abundance of each analyte in a plurality of analytes measured for each capture spot in the set of capture spots from the tissue sample;
B) determining a current iteration of a plurality of proposed cell types that is set to a maximum number of proposed cell types using the respective abundance of each analyte in the plurality of analytes measured for each capture spot in the set of capture spots from the tissue sample, wherein each respective cell type in the current iteration of the plurality of proposed cell types has an abundance value for each analyte in the plurality of analytes;
C) when the current iteration of the plurality of proposed cell types exceeds a minimum number of proposed cell types, performing a procedure comprising:
determining a respective distance metric between each proposed cell type in the current iteration of the plurality of proposed cell types based on the abundance value for each analyte in the plurality of analytes for each proposed cell type in the current iteration of the plurality of proposed cell types, and
reforming the current iteration of the plurality of proposed cell types by merging a first proposed cell type and a second proposed cell type having a smallest distance metric among all unique pairs of proposed cell types in the current iteration of the plurality of proposed cell types;
D) repeating the procedure C) until the current iteration of the plurality of proposed cell types matches the minimum number of proposed cell types; and
E) determining output information, for each respective current iteration of the plurality of proposed cell types,
for each respective proposed cell type in the respective current iteration of the plurality of proposed cell types,
for each respective capture spot in the set of capture spots, providing a respective proportion of cells in the respective capture spot having the respective proposed cell type.

47. A computer readable storage medium storing one or more programs, the one or more programs comprising instructions, which when executed by an electronic device with one or more processors and a memory cause the electronic device to determine cell type by a method comprising:
A) obtaining, in electronic form, input information for a set of capture spots, the input information comprising:
for each capture spot in the set of capture spots, a corresponding position in an image of a tissue sample, and
a respective abundance of each analyte in a plurality of analytes measured for each capture spot in the set of capture spots from the tissue sample;
B) determining a current iteration of a plurality of proposed cell types that is set to a maximum number of proposed cell types using the respective abundance of each analyte in the plurality of analytes measured for each capture spot in the set of capture spots from the tissue sample, wherein each respective cell type in the current iteration of the plurality of proposed cell types has an abundance value for each analyte in the plurality of analytes;
C) when the current iteration of the plurality of proposed cell types exceeds a minimum number of proposed cell types, performing a procedure comprising:
determining a respective distance metric between each proposed cell type in the current iteration of the plurality of proposed cell types based on the abundance value for each analyte in the plurality of analytes for each proposed cell type in the current iteration of the plurality of proposed cell types, and
reforming the current iteration of the plurality of proposed cell types by merging a first proposed cell type and a second proposed cell type having a smallest distance metric among all unique pairs of proposed cell types in the current iteration of the plurality of proposed cell types;
D) repeating the procedure C) until the current iteration of the plurality of proposed cell types matches the minimum number of proposed cell types; and
E) determining output information, for each respective current iteration of the plurality of proposed cell types,
for each respective proposed cell type in the respective current iteration of the plurality of proposed cell types,
for each respective capture spot in the set of capture spots, providing a respective proportion of cells in the respective capture spot having the respective proposed cell type.

48. The method of claim 1, wherein the determining B) comprises:
obtaining a matrix dimensioned by a plurality of objects and a plurality of terms, wherein:
each respective object in the plurality of objects represents a corresponding capture spot in the set of capture spots,
each respective term in the plurality of terms represents a corresponding analyte in the plurality of analytes, and
the obtaining utilizes natural language processing to populate, for each respective object in the plurality of objects and for each respective term in the plurality of terms, a corresponding abundance for the respective analyte in the tissue sample, or a representation thereof, measured at the respective capture spot, wherein the natural language processing comprises a generative statistical model that is further refined by a variational expectation-maximization or Markov chain Monte Carlo procedure.

49. (canceled)
